(19) United States
(12) Patent Application Publication
(10) Pub. No.: US 2026/0177998 A1
(43) Pub. Date: Jun. 25, 2026

(54) SYSTEM AND METHOD FOR OPERATING A DEVICE TO DISPENSE A VOLATILE COMPOSITION IN AN AREA

(71) Applicant: The Procter & Gamble Company, Cincinnati, OH (US)

(72) Inventors: Matthew Stephen BAUER, Loveland, OH (US); Alina Schroeder ROBINSON, Silverton, OH (US); Cody Dylan LEWIS, Lawrenceburg, IN (US); Ronald David TURNER, Cincinnati, OH (US); Matthew Lloyd BARKER, Mason, OH (US)

(21) Appl. No.: 19/424,309

(22) Filed: Dec. 18, 2025

Related U.S. Application Data
(60) Provisional application No. 63/736,899, filed on Dec. 20, 2024.

(51) Int. Cl. G05B 13/04; A01M 1/20; A61L 9/03; A61L 9/12

(52) U.S. Cl. CPC G05B 13/048; A01M 1/2061; A61L 9/035; A61L 9/037; A61L 9/12; A61L 9/122; A61L 2209/111; A61L 2209/131; A61L 2209/133

(57) ABSTRACT

A method may be presented that may include receiving first data from a sensor in an area and determining an awareness pattern in the area based on the first data. The awareness pattern may include low awareness periods and high awareness periods. A value of a characteristic of the first data or the awareness pattern may be determined. When the value of the characteristic is not within the predetermined value range, the method may include transmitting a first signal to the device to operate in a manual mode. When the value of the characteristic is within the predetermined value range, the method may include determining a predicted future low awareness period and transmitting a second signal to the device such that the device operates in an automatic mode during the predicted future low awareness period. A system may also be provided that performs the method.

### TECHNICAL FIELD

The present disclosure relates to a method of operating a device to dispense a volatile composition in an area based on data indicating an awareness of a subject in the area.

### BACKGROUND

Volatile composition dispensers exist for delivering various volatile compositions, such as freshening compositions, into the air. Such volatile composition dispensers may, for example, take the form of a wick-based electrical dispenser having one or more heaters to assist with volatizing the volatile composition into the air. Consumers desire for the volatile composition dispenser to provide noticeability and longevity without having to frequently replenish the volatile composition. However, conventional volatile composition dispensers typically operate in a constant manual mode, where the volatile composition is dispensed at a constant rate, regardless of subject occupancy or awareness of the dispensed volatile composition. Thus, these conventional volatile composition dispensers do not optimize the noticeability and longevity of the volatile composition within the dispenser.

### SUMMARY

In one aspect, the invention may feature, in general, a method for operating a device. The method may include receiving, at a processor, first data over a first time period from a sensor positioned in an area. The method may also include determining, with the processor, second data that may indicate an awareness pattern in the area over the first time period based on the first data. The awareness pattern may include one or more low awareness periods with a low level of awareness in the area and one or more high awareness periods with a high level of awareness in the area. The method may also include determining, with the processor, third data that may indicate a value of a characteristic of the first data or the awareness pattern. The method may also include determining, with the processor, whether the value of the characteristic may be within a predetermined value range. The method also includes transmitting, from the processor, a first signal to the device such that the device operates in a manual mode; when the value of the characteristic is not within the predetermined value range. When the value of the characteristic is within the predetermined value range, the method includes the steps of determining, with the processor, a predicted future low awareness period over a second time period after the first time period based on the awareness pattern of the second data, and transmitting, from the processor, a second signal to the device such that the device operates in an automatic mode during the predicted future low awareness period.

In another aspect, the invention features, in general, the steps of the method of the first aspect but further includes that the characteristic of the first data or awareness pattern may be one or more of:
a) a level of continuity of the first data over the first time period,
b) a difference between a maximum value and a minimum value of the first data over the first time period,
c) a difference between a first distance between a first pair of consecutive low awareness periods of the awareness pattern and a second distance between a second pair of consecutive low awareness periods of the awareness pattern,
d) a duration of one of the low awareness periods or the high awareness periods, and
e) a number of the one or more low awareness periods or the one or more high awareness periods within a fixed time interval.

In another aspect, the invention features, in general, a system that includes a device and a sensor positioned in an area and configured to measure first data. The system also includes a processor communicatively coupled with the sensor and the device and configured to receive the first data from the sensor over a first time period. The processor may be configured to determine second data that indicates an awareness pattern in the area over the first time period based on the first data. The awareness pattern includes one or more low awareness periods with a low level of awareness in the area and one or more high awareness periods with a high level of awareness in the area. The processor may be configured to determine third data that indicates a value of a characteristic of the first data or the awareness pattern. The processor may be configured to determine whether the value of the characteristic may be within a predetermined value range. When the value of the characteristic is not within the predetermined value range, the processor may be configured to transmit a first signal to the device such that the device may operate in a manual mode. When the value of the characteristic is within the predetermined value range, the processor may be configured to determine a predicted future low awareness period over a second time period after the first time period based on the awareness pattern of the second data, and transmit a second signal to the device such that the device may operate in an automatic mode during the predicted future low awareness period.

### BRIEF DESCRIPTION OF THE DRAWINGS

FIG. 1 is a partially fragmented schematic front view showing a volatile composition dispenser comprising two delivery engines in the form of wicks.

FIG. 2 is a partially fragmented schematic side view of the device shown in FIG. 1.

FIG. 3 is a schematic top view of the device shown in FIG. 1.

FIG. 4A is a block diagram of the device shown in FIG. 1 positioned in an area.

FIG. 4B is a block diagram of a plurality of the devices shown in FIG. 4A each positioned in a respective area of a building.

FIG. 5 is a schematic, exploded view of a volatile composition dispenser having a cartridge with a membrane as a delivery engine.

FIG. 6 is a schematic, exploded view of the cartridge of FIG. 5.

FIG. 7 is a schematic, perspective view of a printed circuit board that may be used to control the volatile composition dispenser shown in FIGS. 1 to 4A, along with heaters and a plug that are attached thereto.

FIG. 8 is a flowchart that depicts one or more steps of a method for operating a volatile composition dispenser.

FIG. 9A is a plot that indicates a signal transmitted from the controller to the evaporative assistance element in a conventional volatile composition dispenser.

FIG. 9B is a plot that indicates a signal transmitted from the controller to the evaporative assistance element of FIG. 7 based on an awareness pattern.

FIG. 9C is a schematic view that indicates a time window of sensor data captured by the sensor of FIG. 7 and smoothed sensor data over the time window.

FIG. 9D is a plot that indicates sensor data captured by the sensor of FIG. 7 over time.

FIGS. 9E and 9F are plots that indicate the sensor data of FIG. 9D after suppressing sensor data above a threshold value.

FIG. 9G is a plot that indicates the suppressed sensor data of FIG. 9F after performing a smoothing operation.

FIG. 9H is a plot that indicates the smoothed sensor data of FIG. 9G after performing a normalization operation.

FIG. 9I is a plot that indicates an awareness pattern that is the normalized sensor data of FIG. 9H after performing a digitizing operation.

FIG. 10A is a plot that indicates an awareness pattern including a plurality of low and high awareness periods over a first time period and a plurality of predicted future low and high awareness periods over a second time period.

FIG. 10B is a plot that indicates a center of the plurality of low awareness periods and a center of the plurality of predicted future low awareness periods of FIG. 10A.

FIG. 11 is a block diagram that illustrates a computer system upon which an aspect of the disclosure may be implemented.

FIG. 12 is a block diagram that illustrates a chip set upon which an aspect of the disclosure may be implemented.

FIG. 13 is a block diagram that illustrates a mobile terminal upon which an aspect of the disclosure may be implemented.

### DETAILED DESCRIPTION

The methods and systems disclosed herein may solve the problem of reduced noticeability and longevity of volatile compositions and thus may reduce the frequency at which the volatile composition needs to be replenished in volatile composition dispensers.

Attempts have been made to address this problem of reduced longevity of volatile compositions in dispensers. For example, light sensors have been introduced to instantaneously measure an amount of light in an area and use this as an indication of subject occupancy or awareness in the area. The device is activated if the instantaneously measured light exceeds a light threshold. Similarly, motion sensors have been used to instantaneously measure motion in the area and use this as an indication of subject occupancy and awareness in the area. The device is then activated based on the instantaneously measured motion.

It was recognized that these conventional methods have noticeable drawbacks and may not effectively solve the problem of reduced noticeability and longevity of volatile compositions in the dispensers. For example, volatile composition dispensers typically feature a time-to-action (e.g., about 15 minutes) between when the volatile composition dispenser is activated to when the volatized composition is detectable. Consequently, even if the conventional methods employ sensors to instantaneously measure light or motion data and use this sensor data to activate the volatile composition device, by the time the volatized composition is detectable, the subject may not still be in the area. Additionally, in another example, instantaneous light or motion conditions may change frequently in many scenarios and for reasons that may not correlate to a general period of subject occupancy or awareness in an area. For example, if a subject goes to the bathroom in the middle of the night, this action may trigger light and motion sensors yet would not indicate a sufficiently long awareness period of the subject in the area to detect the volatized composition. It was recognized that light and dark conditions in a home may vary widely for a variety of factors including short term occupancy activities, use of blinds, cloud cover, and relocation of devices. Further, if a device is allowed to react to highly variable light conditions within most homes, the variability in experience could degrade both the overall scent experience and expectations based upon claims of device longevity.

Therefore, it was recognized that there is a need to establish an awareness pattern or long-term trend to accurately predict future periods of subject occupancy and awareness in an area and to ensure that the duration of such periods may be sufficiently reliable to make effective operational decisions of the volatile composition dispenser. For example, such long-term trends may be used to classify scenes where a circadian pattern is present.

Embodiments of the present disclosure are directed to a volatile composition dispenser and method of delivering a volatile composition into the air using a volatile composition dispenser. The volatile composition dispenser may be configured to deliver a volatile composition into the air with increased longevity of the volatile composition contained within a reservoir. It has been found that the method disclosed herein may also enhance consumer noticeability of the volatile composition over time. In particular, an awareness pattern may be determined that indicates one or more time periods of low awareness and one or more time periods of high awareness in an area. This awareness pattern may then be used to determine when to activate the volatile composition dispenser.

The method disclosed herein may encompass a broad set of algorithms including, but not limited to, a Time Series (present execution), neural networks (NN), Logistic Regression, Support Vector Machine, Gradient Boosted Trees, Random Forest, Recurrent Neural Networks, and general machine learning (ML) approaches for classifying low vs. high occupancy awareness periods, allowing for singular or a plurality of models used in parallel or sequence to establish a high accuracy prediction. Each of these models may be used to replace the hand engineered feature extraction steps of the method disclosed herein to determine a midpoint of each low awareness/high awareness period and a predicted duration between midpoints, provided labeled data is used to train the models and that they can be computed given the available CPU resources on the device. A final prediction of the future night period may remain a simple downstream math function in the presence of output from these different model structures. The method disclosed herein may allow for a single or combination of multiple model architectures, paired with manual features. Examples of such a broad set of algorithms disclosed in other contexts may include, but may not be limited to, European Patent No. 3513927 B2, U.S. Pat. No. 11,978,207 B2, and Canadian Patent No. 30159492 C, which are each incorporated herein by reference.

An additional consideration is that an alternative adaptive algorithm may be engineered that may allow the per-user device to self-learn what a low awareness and high awareness period could look like for a given consumer. For example, if a source of reinforcement data were available, such as information about user geolocation, user utilization of a mobile app, user response to a push notification, then an algorithm may redefine the low awareness period to include data which is significantly more noisy or atypical based upon the reinforcement knowledge. Further, a federated cloud learning model may pool such data and pattern insights across multiple households and deploy model changes based upon the federated knowledge base. For example, if a macro trend in time of low awareness period onset were observed across households, the model may be retrained to understand the difference between low light due to user specific actions vs. seasonal daylight changes and/or in specific geolocations.

The term “volatile compositions,” as used herein, may refer to a material that may comprise a vaporizable material. The term “volatile compositions,” thus may include (but is not limited to) compositions that are comprised entirely of a single volatile material. The terms “volatile materials,” “aroma,” “fragrance,” and “scents,” as used herein, may include, but are not limited to, pleasant or savory smells, and, thus, also encompass materials that function as insecticides, air fresheners, deodorants, aromacology, aromatherapy, insecticides, or any other material that acts to condition, modify, or otherwise charge the atmosphere or to modify the environment. It should be understood that certain volatile compositions may include, but not limited to, perfumes, aromatic materials, and scented materials, may often comprise one or more volatile materials (which may form a unique and/or discrete unit comprised of a collection of volatile materials). It should be understood that the term “volatile composition” may refer to compositions that have at least one volatile component, and it may not be necessary for all of the component materials of the volatile composition to be volatile. The volatile compositions described herein may, thus, also have non-volatile components. It should also be understood that when the volatile compositions are described herein as being “emitted,” this may refer to the volatilization of the volatile components thereof and may not require that the non-volatile components thereof be emitted. The volatile compositions of interest herein may be in any suitable form including, but not limited to, solids, liquids, gels, encapsulates, and combinations thereof.

It is contemplated that the volatile composition dispenser may be configured for use in a variety of applications to deliver the volatile composition to the air and/or ultimately to a surface. The volatile composition dispenser may be configured in various ways.

For example, the volatile composition dispenser may be configured as an electrical wall plug or battery-operated volatile composition dispenser that may have a housing, a reservoir containing a volatile composition, a delivery engine that may be used to transport the volatile composition to an evaporative surface, and an evaporative assistance element to assist with the volatilization of the volatile composition from the evaporative surface. The evaporative assistance element may be placed adjacent to the evaporative surface.

The reservoirs may comprise any suitable type of container and may be made of any suitable material. Suitable materials for the reservoirs may include, but are not limited to, glass and plastic. The reservoirs may comprise any type of container that is suitable for holding volatile compositions.

The reservoirs may be part of the housing, or they may be separate components that are removably joined to a portion of the volatile composition dispenser such as the housing. It is also possible for a single reservoir to hold more than one type of volatile material. Such a reservoir may, for instance, have two or more compartments for volatile materials.

The delivery engine may comprise the evaporative surface. In such a configuration, the delivery engine may be placed next to one or more evaporative assistance elements, such as a heater or fan to volatilize the volatile composition into the air. The evaporative assistance elements may surround or at least partially surround the evaporative surface.

Instead of evaporating the volatile composition from an evaporative surface of the delivery engine, the delivery engine may transport the volatile composition to a separate evaporative surface. The evaporative surface may be configured as a porous or semi-porous substrate, a bowl or plate, including a plastic, glass, or metal bowl or plate, and combinations thereof.

The delivery engine may be configured in various ways. For example, the delivery engine may be in the form of a wick, membrane, gel, wax, porous or semi-porous substrate, including a felt pad. In a volatile composition dispenser comprising more than one delivery engine associated with the same or different reservoirs, the delivery engines may be the same or may be different.

If the volatile composition dispenser utilizes a wick as a delivery engine, the wick may be configured to have various different shapes and sizes. For example, the wick may have a cylindrical or an elongate cube shape. The wick may be defined by a length and a diameter or width, depending on the shape. The wick may have various lengths. For example, the length of the wick may be in the range of about 1 millimeter (“mm”) to about 100 mm, or from about 5 mm to about 75 mm, or from about 10 mm to about 50 mm. The wick may have various diameters or widths. For example, diameter or width of the wick may be at least 1 mm, or at least 2 mm, or at least 3 mm, or at least 4 mm. A wick may exhibit a density. The wick density may be in the range of about 0.100 grams/cm³ (“g/cc”) to about 1.0 g/cc.

A wick may comprise a porous or semi-porous substrate. The wick may be composed of various materials and methods of construction, including, but not limited to, bundled fibers which are compressed and/or formed into various shapes via overwrap (such as a non-woven sheet over-wrap) or made of sintered plastics such as PE, HDPE, or other polyolefins. The wick may be made from a plastic material such as polyethylene or a polyethylene blend.

The evaporative assistance element may be used to assist with the evaporation of a volatile composition from the evaporative surface. For example, the evaporative assistance element may be selected from the group consisting of a heater, a fan, an agitation member or agitator that cause vibration, both powered agitator and manual agitator, or combinations thereof. The evaporative assistance element may also include a heating element to heat the liquid volatile composition, a chemical constituent to speed evaporation or evaporation rates, use of a chemically heated membrane to provide increased evaporation via exothermic reaction, or synergistic combinations thereof. The evaporative assistance element may also increase the amount of surface area of a delivery engine exposed to the evaporative assistance element, may cause a pressure gradient, rheostate, etc.

A volatile composition dispenser that may have an evaporative assistance element in the form of a heater may be configured to heat the evaporative surface to various temperatures. For example, the volatile composition dispenser may be configured such that the heater raises the temperature of the evaporative surface to a temperature of about 30° C. to about 150° C. The heater(s) may comprise any suitable type of heater and may be located in any suitable location in or relative to the volatile composition dispenser. The evaporative assistance element may surround or at least partially surround the evaporative surface.

The term “awareness pattern”, as used herein, may refer to a pattern that indicates a level of awareness of a human being in an area over time. The awareness pattern may include one or more low awareness periods with a low level of awareness in the area and one or more high awareness periods with a high level of awareness in the area. For example, if a person is physically present in the area but not cognitively aware of their surroundings (e.g., sleeping) this may indicate a low level of awareness. In another example, if a person is physically absent from the area such that no person is present in the area, this may indicate a low level of awareness. In another example, if a person is moving in the area (e.g., walking) in a manner other than typical movements associated with sleep (e.g., turning from side to side on a bed) this may indicate a high level of awareness. In yet another example, if a person is not moving around the area (e.g., sitting, lying down, etc.) but is engaged in some activity that indicates cognitive awareness (e.g., reading a book, watching TV, using a smartphone, talking on the phone, talking with another person sitting in the area, etc.) this may indicate a high level of awareness. The awareness pattern may follow a regular pattern, such as a regular high awareness period in the area (e.g., between 7 am and 9 pm) when the person is present in the area and engaged in some activity indicating cognitive awareness and a regular low awareness period in the area (e.g., between 9 pm and 7 am) when the person is sleeping. In yet another example, if the person is absent from the area for an extended time (e.g., on vacation), then the awareness pattern may indicate a low awareness period with a constant low level of awareness over the extended time period.

The term “characteristic of data”, as used herein, may refer to a parameter that may be used to describe one or more properties of the data. In one example, where the data is recorded over time the characteristic may be a level of continuity of the data over time (e.g., where a high value of the characteristic may indicate little to no discontinuity in the data over time and thus a high level of continuity in the data over time). In yet another example, the characteristic may be a range or difference between a maximum value and a minimum value of the data over time (e.g., where a higher value of the characteristic may represent a larger range between the maximum and minimum values and a lower value of the characteristic may represent a smaller range between the maximum and minimum values).

The term “characteristic of an awareness pattern”, as used herein, may refer to a parameter that may be used to describe one or more properties of the awareness pattern with a plurality of low awareness periods and a plurality of high awareness periods. In one example, the characteristic may indicate a level of regularity to the awareness pattern. For example, where the awareness pattern features a first pair of consecutive low awareness periods separated by a first distance and a second pair of consecutive low awareness periods separated by a second distance, the characteristic may be a difference between the first and second distance. In this example, a low value of the difference may indicate a high regularity to the awareness pattern whereas a high value of the difference may indicate a low regularity to the awareness pattern. In another example, the characteristic may be a value of a duration of one of the low awareness periods or the high awareness periods of the awareness pattern. In yet another example, the characteristic may be a number of the low awareness periods and/or high awareness periods which occur within a fixed time interval (e.g., such as 24 hours).

The term “value of a characteristic of data”, as used herein, may refer to a numerical quantity of the characteristic of the data.

The term “data that indicates an awareness pattern”, as used herein, may refer to data that may be the awareness pattern or may be used to determine the awareness pattern. For example, where the data is sensor data obtained over time, a mathematical operation (e.g., smoothing) may be performed on this sensor data to obtain the awareness pattern over time (e.g., so that the low levels of awareness of the awareness pattern may be more defined and clear).

The term “data that indicates a value of a characteristic of data”, as used herein, may refer to data that may be the value of the characteristic of the data or may be used to determine the value of the characteristic of the data. For example, where the characteristic is a difference between a maximum value and a minimum value over time, the data may be the maximum value and the minimum value which may be used to determine the difference therebetween and thus may indicate the characteristic of the data.

The term “data that indicates a value of a characteristic of an awareness pattern”, as used herein, may refer to data that may be the value of the characteristic of the awareness pattern or may be used to determine the value of the characteristic of the awareness pattern. For example, where the characteristic may be a difference between a first distance between a first pair of consecutive low awareness patterns and a second distance between a second pair of consecutive low awareness patterns, the data may be the awareness pattern including the low awareness patterns which may be used to determine the first distance, the second distance and the difference therebetween. Thus, the data may indicate the characteristic of the awareness pattern.

The term “low level of awareness”, as used herein, may refer to a lack of cognitive awareness of a human being in an area. In one example, if no human being is present in the area, this may indicate a low level of awareness. In another example, if a human being is present in the area but not cognitively aware of their surroundings in the area (e.g., sleeping) this may indicate a low level of awareness.

The term “high level of awareness”, as used herein, may refer to a level of awareness of a human being in an area where a human being is physically present and engaged in some type of activity that indicates cognitive awareness in the area. In one example, if a person is moving in the area (e.g., walking, cooking, etc.) in a manner other than typical movements associated with sleep (e.g., turning from side to side in a bed) this may indicate a high level of awareness. In another example, if a person is not moving within the area but is engaged in some type of activity that indicates cognitive awareness (e.g., reading a book, watching TV, using a smartphone, talking on the phone, talking with another person in the area, etc.) this may indicate a high level of awareness.

The term “low awareness period”, as used herein, may refer to a time period of the awareness period that corresponds to a low level of awareness.

The term “consecutive low awareness periods”, as used herein, may refer to two low awareness periods that are consecutive in time along the awareness pattern with no low awareness period therebetween. In one example, a high awareness period of the awareness pattern may occur between the consecutive low awareness periods.

The term “manual mode”, as used herein, may refer to a mode of the volatile composition dispenser which operates in accordance with a manual setting on the volatile composition dispenser (e.g., low, medium, high). In one example, if the manual setting is set to high, then when the manual mode of the volatile composition dispenser is activated, the volatile composition dispenser may dispense a high amount of volatile composition from a reservoir within the volatile composition dispenser per unit time. In another example, if the manual setting is set to low, then when the manual mode of the volatile composition dispenser is activated, the volatile composition dispenser may dispense a low amount of volatile composition from the reservoir within the volatile composition dispenser per unit time.

The term “automatic mode”, as used herein, may refer to a mode of the volatile composition dispenser which may be automatically activated by a processor based on the awareness pattern. In one example, the automatic mode may be activated by the processor during a predicted future low awareness period which may be determined by the processor using the awareness pattern. In one example, the automatic mode may include a low mode where a low amount of volatile composition from a reservoir within the volatile composition dispenser is dispensed per unit time. In yet another example, the automatic mode may include a high mode where a high amount of volatile composition from the reservoir within the volatile composition dispenser is dispensed per unit time.

The term “determining, with a processor, a second data based on a first data”, as used herein, may refer to the second data that is determined using the first data. For example, where the first data is sensor data including incremental sensor data values that are obtained over respective time increments, the first data may be combined to determine second data that may include each of the sensor data values over a time period that encompasses multiple time increments.

The term “smoothed curve”, as used herein, may refer to a curve with one or more peaks and one or more valleys that may be modified such that the one or more peaks and/or the one or more valleys are more clearly defined. In one example, the smoothed curve may increase a magnitude of the slope of one or more portions of the curve between one or more peaks and one or more valleys such that the peaks and valleys are more clearly defined.

The term “normalizing a value of the smoothed curve”, as used herein, may refer to a range of values of a curve between a minimum value and a maximum value are rescaled such that the minimum value of the normalized curve may correspond to 0 and the maximum value of the normalized curve may correspond to 1.

The term “digitizing the smoothed curve”, as used herein, may refer to the values of the curve including one or more peaks and one or more valleys are modified such that the one or more values of the curve (e.g., above a threshold value) are assigned a value of 1 and all other portions of the curve are assigned a value of 0.

### System For Volatizing a Composition

As will be discussed in more detail below, the volatile composition dispenser may include a control system to control the evaporative assistance element.

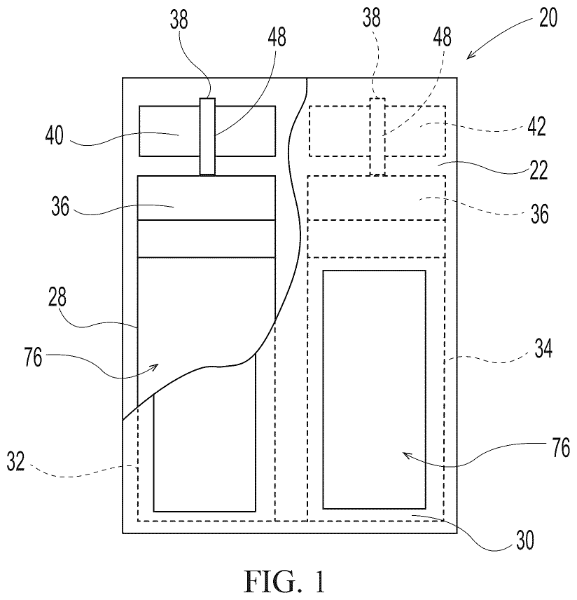

With reference to FIGS. 1 to 4A, the volatile composition dispenser or device 20 may take the form of an electrical wall plug volatile composition dispenser. The volatile composition dispenser 20 may include a housing 22, and the housing 22 is supported on an electrical outlet 24 by a power source 26 that is at least indirectly joined to the housing 22. The volatile composition dispenser 20 may further comprise at least one reservoir, shown as reservoirs 28 and 30 for illustrative purposes, for containing the volatile compositions, respectively. The housing 22 may serve as a holder for the reservoir(s) 28 and 30 and any of the other components of the volatile composition dispenser 20. The volatile composition dispenser 20 may comprise one or more delivery engines 38, shown as wicks in FIGS. 1 to 4A for illustrative purposes only, that may extend into each reservoir 28, 30 at one end of the delivery engine and may have an evaporative surface 48 at the opposite end. The volatile composition dispenser may include one or more evaporative assistance elements 40, 42, such as a heater as shown in FIGS. 1 to 4A for illustrative purposes only, for assisting with the evaporation of the volatile compositions from the evaporative surfaces 48. The reservoirs 28 and 30 may contain a first volatile composition 32 and a second volatile composition 34.

Some parts of the volatile composition dispenser may be joined together to form a cartridge 76. For example, the reservoir(s), delivery engine(s), evaporative surface(s), and/or evaporative assistance element(s) may be joined together as one or more cartridges 76. With reference to FIG. 1, a reservoir 28 or 30, delivery engine 38, and evaporative surface 48 may be connected together to form a cartridge 76. The volatile composition dispenser shown in FIG. 1 may include two cartridges 76, for example.

The cartridges or reservoirs may be replaceable to provide a reservoir with a new, different, or replacement volatile composition. Or, the reservoirs may be refillable and reused in the volatile composition dispenser in a new total emission program.

The heater(s), such as heaters 40 and 42 shown in FIGS. 1 to 4A for illustrative purposes only, may comprise heating elements that are in the form of circular rings that at least partially surround the wicks protruding from the bottles of the volatile compositions.

The reservoir(s) may comprise a seal 36, such as shown in FIG. 1, for containing the volatile composition. The volatile composition dispenser 20 and/or the reservoirs 28 and 30 may further comprise an additional seal for covering the wick 38 when the volatile composition is not being emitted.

While FIG. 1 illustrates two reservoirs, two evaporative assistance elements, and two delivery engines, it is to be appreciated that a volatile composition dispenser may include one, two, three, or more reservoirs. Each reservoir in a volatile composition dispenser may include a separate delivery engine. A single evaporative assistance element may be used for one or more evaporative surfaces or each evaporative surface may be adjacent to a unique evaporative assistance element. If the volatile composition dispenser includes more than one reservoir, each reservoir may contain a different volatile composition or may contain the same volatile composition.

While it is shown in FIGS. 1 to 4A that the volatile composition dispenser 20 may include two reservoirs, it is to be appreciated that the volatile composition dispenser may comprise one or more than one reservoir. If one reservoir is present, the volatile composition dispenser may include one, two, or more than two delivery engines that are each in fluid communication with the one reservoir and one, two, or more evaporative surfaces that are in fluid communication with the delivery engines. In such a configuration, the volatile composition dispenser may include one or more evaporative assistance elements. If more than one delivery engine is in fluid communication with a single reservoir, than each delivery engine may be used to volatilize the same volatile composition. This configuration may allow for each delivery engine, such as a wick, to have an extended period where the evaporative assistance element is either delivering low energy or is OFF, giving each delivery engine time for the volatile composition to drain and potentially unclog from the delivery engine. Such a configuration may be particularly useful where the delivery engines are in the form of wicks, which may suffer from wick-clogging of components of volatile compositions.

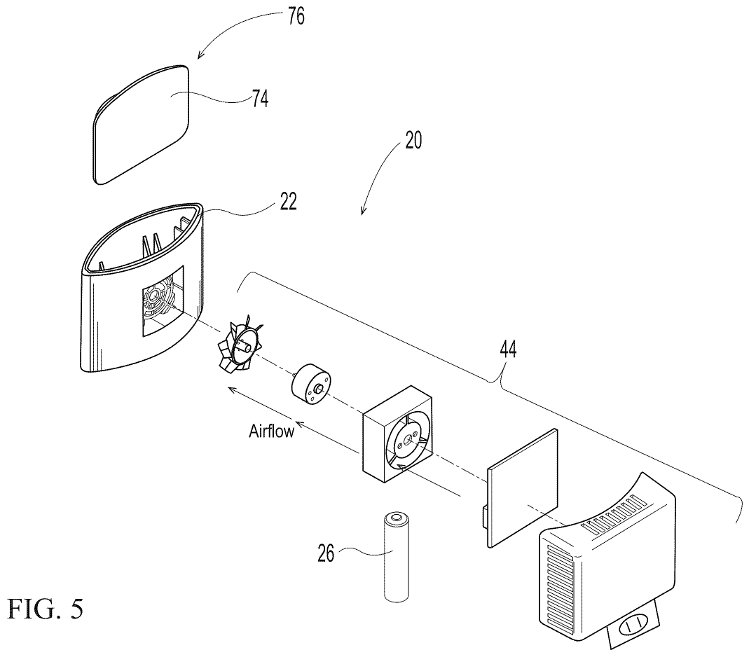

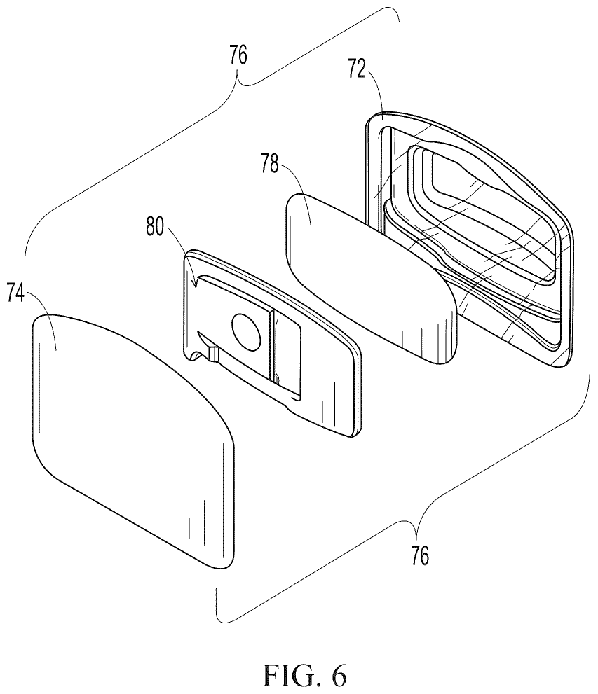

Instead of a wick, the delivery engine may be comprised of a breathable membrane. With reference to FIGS. 5 and 6, the volatile composition dispenser 20 may comprise a cartridge 76. The cartridge 76 may include a liquid reservoir 72 for containing a volatile composition and a delivery engine 74 in the form of a breathable membrane enclosing the liquid reservoir 72, such as disclosed in U.S. Pat. Nos. 8,709,337 and 8,931,711. The volatile composition dispenser 20 may also include an evaporative assistance element 44 in the form of a fan as shown in FIG. 6 for exemplary purposes only. As used herein, a breathable membrane may be a vapor permeable membrane that prevents free flow of liquid out of the membrane, thus addressing leakage problems.

Suitable breathable membranes may include, but are not limited to, UHMWPE-type membrane optionally filled with silica as described in U.S. Pat. No. 7,498,369. Such UHMWPE membranes may include Daramic™ V5, available from Daramic, Solupor®, available from DSM (Netherlands), and Teslin™ SP1100HD, available from PPG Industries, and combinations thereof. Other suitable breathable membranes may include any permeable polymeric, thermoplastic, or thermoset material, including acetal, acrylic, cellulosic, fluoroplastic, polyamide, polyester, polyvinyl, polyolefin, styrenic, etc, alone, co-extruded, woven or non-woven, mixed or in combination with elastomers, rubber, solids, silicas, or combinations thereof. Also suitable are Hytrel™ available from Dupont or Lotryl™ available from Arkema. The delivery engine 74, such as shown in FIG. 6, may also include a rupturable substrate 78 that seals the volatile composition in the liquid reservoir until a rupture mechanism 80 is engaged to when the volatile composition dispenser is to be used by the consumer. When the consumer is ready to use the volatile composition dispenser, the consumer may rupture the rupturable substrate 78 with the rupture mechanism 80, which allows the volatile composition in the liquid reservoir 72 to contact the breathable membrane. Alternatively or additionally, the delivery engine 74 may have an outer vapor impermeable layer (e.g., a foil layer) that can be removed before use.

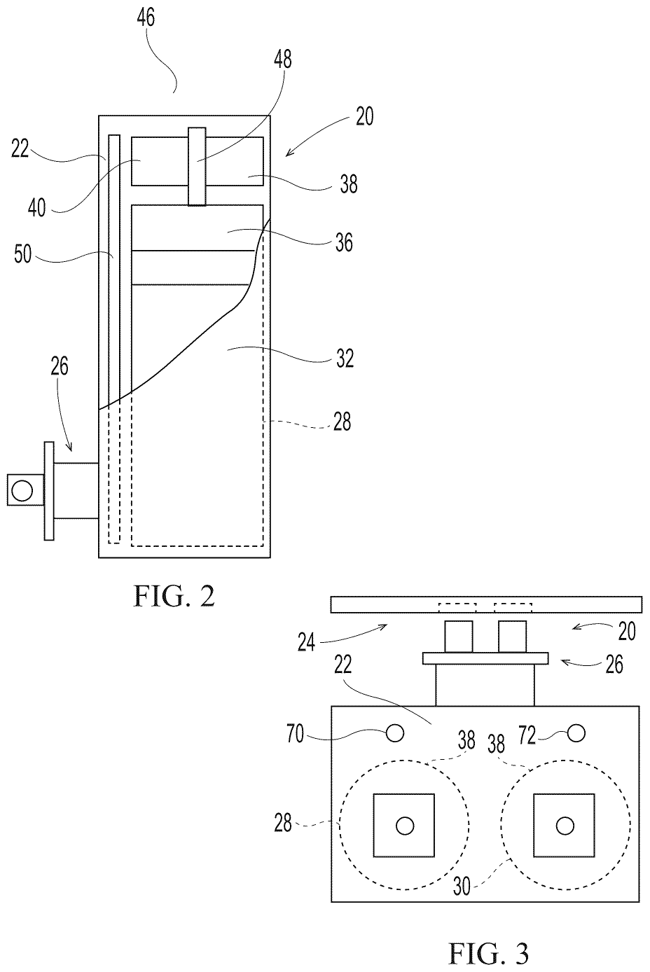

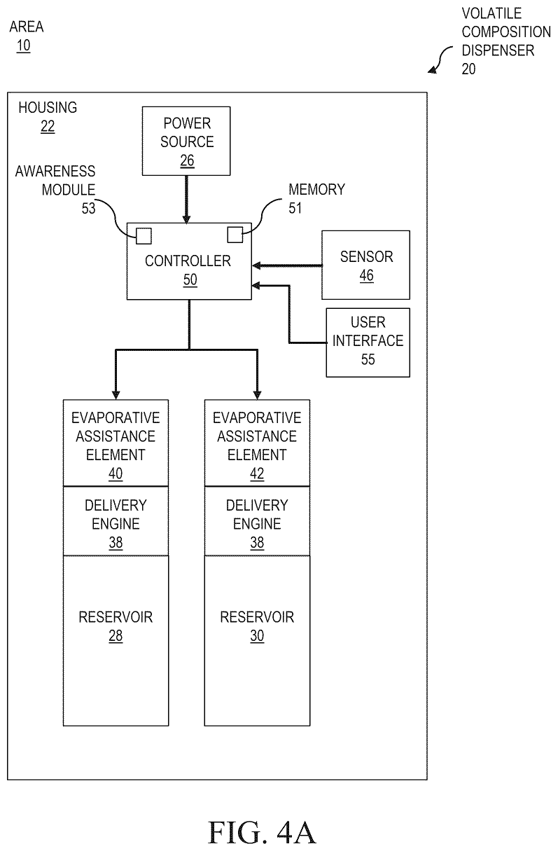

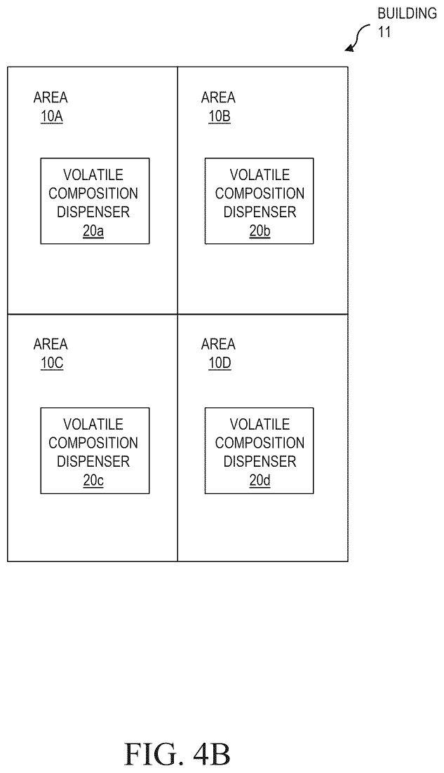

With reference to FIGS. 2 and 4A, the volatile composition dispenser 20 may include a processor or controller 50 that may change the volatile composition being emitted by the volatile composition dispenser 20. The controller 50 may be communicatively coupled with the evaporative assistance elements 40, 42 (e.g., heaters) and may be configured to transmit a signal to the evaporative assistance elements 40, 42 to adjust the temperature of the delivery engine(s) 38 and consequently adjust the rate of volatilization of the volatile composition from the dispenser 20. Similarly, the controller 50 may be communicatively coupled to the evaporative assistance element 44 (e.g., fan) in the examples of the volatile composition dispenser 20 of FIGS. 5 and 6. In one example, the controller 50 may transmit a first signal to the evaporative assistance elements 40, 42 such that the dispenser 20 operates in a manual mode and may transmit a second signal to the evaporative assistance elements 40, 42 such that the dispenser 20 operates in an automatic mode.

The manual mode may be based on a manual setting (e.g., low, medium, high) of a user with a user interface 55 (e.g., switch) on the housing 22. In this example, the setting (e.g., low, medium, high) of the user interface 55 may determine a heating temperature of the evaporative assistance elements 40, 42 (and delivery engine 38) upon receiving the first signal from the controller 50 and consequently may determine a heating temperature of the volatile composition on the delivery engine 38 heated by the evaporative assistance elements 40, 42.

The automatic mode of operation of the dispenser 20 may be based on sensor data received at the controller 50 from a sensor 46. In one example, the sensor 46 may be positioned on an outer surface of the housing 22. However, in other examples, the sensor 46 may be remote from the housing 22 and is in wireless communication with the controller 50. In one example, the sensor 46 may be a light sensor that may be configured to measure light data in an area 10, such as at regular time increments (e.g., 15 minutes). In another example, the sensor 46 may be a motion sensor that may be configured to measure motion in the area 10, such as at regular time increments (e.g., 15 minutes). In one example, the area 10 may be a room, such as a room in a residence or commercial building.

As discussed in more detail of the method herein, in some examples the controller 50 may receive the sensor data from the sensor 46 over a first time period (e.g., between about 10 hours and 5 days). The controller 50 may then determine an awareness pattern over the first time period based on the sensor data that includes one or more low awareness periods (with no or minimal level of subject awareness of the area) and one or more high awareness periods (with a high level of subject awareness of the area). The controller 50 may further predict a future low awareness period over a second time period after the first time period, based on the awareness pattern. The controller 50 may then transmit the second signal to the evaporative assistance elements 40, 42 such that the dispenser 20 operates in the automatic mode during the future low awareness period. In the automatic mode, the evaporative assistance elements 40, 42 may be heated to a low temperature (or zero temperature) upon receiving the second signal from the controller 50. In one example, the temperature of the evaporative assistance elements 40, 42 during the automatic mode may be set by the user using the user interface 55. It was recognized that this may advantageously conserve the volatile composition, since it may minimize and/or eliminate volatilization of the composition during periods of low awareness of the area (when there is no or minimal level of subject awareness of the area).

Although the above examples discuss that the sensor 46 may be a light sensor or a motion sensor, the sensor 46 is not limited to these specific sensors and may include any sensor capable of capturing sensor data that could be utilized as an indication of subject awareness of an area. For example, the sensor 46 may be a camera or imaging device that is configured to capture image data of the area. In this example, upon receiving the sensor data from the camera or imaging device, the controller 50 may process the image data to determine a level of subject awareness of the area. In one example, the controller 50 may process the image data to determine that a subject is in a seated position or a standing position in the area and may interpret this as a high level of subject awareness of the area. In another example, the controller 50 may process the image data to determine that a subject is in a laying down position in the area and may interpret this as a low level of subject awareness of the area. In yet another example, the sensor 46 may be a position sensor, such as a GPS sensor in a smartphone. In this example, the controller 50 may process the sensor data from the GPS sensor to determine a level of subject awareness of the area (e.g., a high level of subject awareness based on a changing location from the GPS sensor). In yet another example, the sensor 46 may indicate a level of activity of an internet network in the area, such as a level of activity of a smartphone and/or a computer connected to the internet network. In this example, the controller 50 may process the sensor data to determine a level of subject awareness of the area (e.g., high level of subject awareness based on a high level of activity of the internet network, etc.).

In another example, although a single sensor 46 is depicted in FIG. 4A, in other examples multiple sensors 46 may be provided and the sensor data from the multiple sensors 46 may be processed by the controller 50 to determine the level of subject awareness in the area. In one example, where a light sensor 46 and image sensor 46 are provided in the area, the controller 50 may determine a high level of subject awareness based on a combination of processing the image data from the imaging sensor 46 that may indicate the subject in a laying down position in conjunction with light sensor data from the light sensor 46 that may indicate a presence of light in the area.

Although FIG. 4A depicts a single dispenser 20 positioned within a single area 10, in other aspects of the disclosure, as shown in FIGS. 4B, a plurality of dispensers 20a, 20b, 20c, 20d may be respectively positioned in a plurality of areas 10a, 10b, 10c, 10d (e.g., rooms) of a building 11 (e.g., residence, commercial business, etc.). In this example, each respective dispenser 20a, 20b, 20c, 20d may feature a respective sensor 46 that separately measures sensor data within each respective area 10a, 10b, 10c, 10d and may further feature a respective controller 50 which may determine a respective awareness pattern in each respective area 10a, 10b, 10c, 10d based on the respective sensor data in that area. Based on these respective awareness patterns, each controller 50 may individually determine whether to activate the manual mode or the automatic mode of each respective dispenser 20a, 20b, 20c, 20d based on the respective awareness pattern in each respective area 10a, 10b, 10c, 10d. In yet another example, although FIG. 4B depicts four areas 10a, 10b, 10c, 10d, the number of areas within the building 11 may be less or more than four areas. Additionally, in yet another example, although FIG. 4B depicts the plurality of areas 10a, 10b, 10c, 10d within a single building 11, in other examples the plurality of areas may be positioned within multiple buildings.

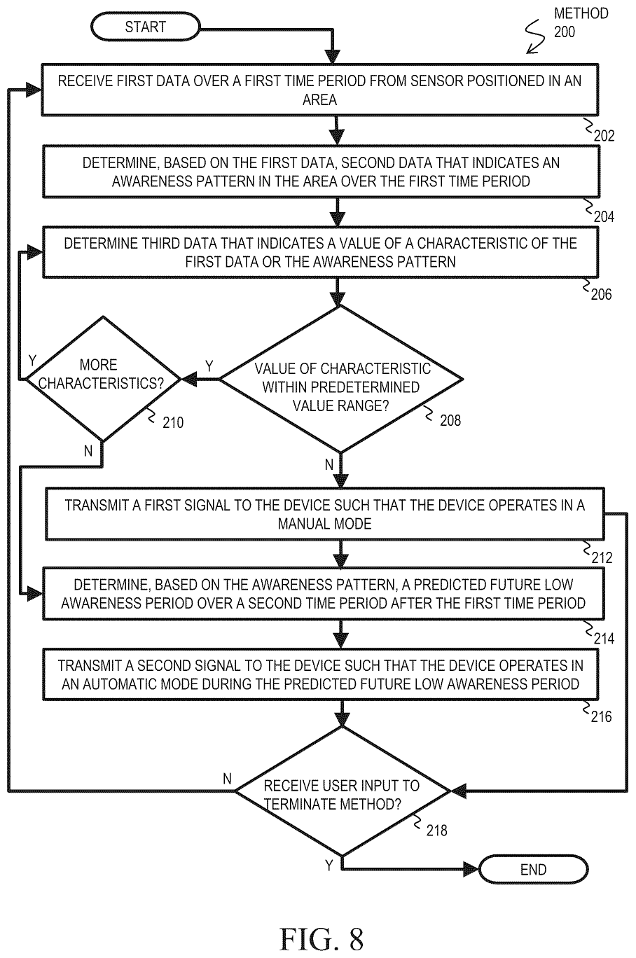

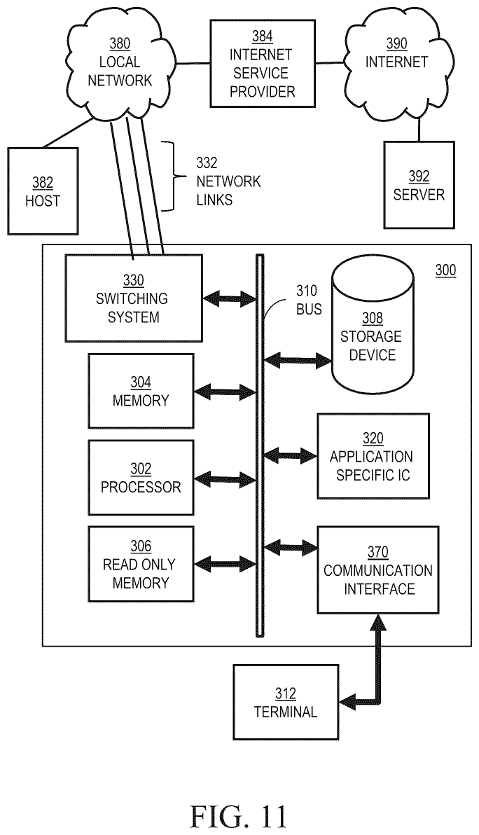

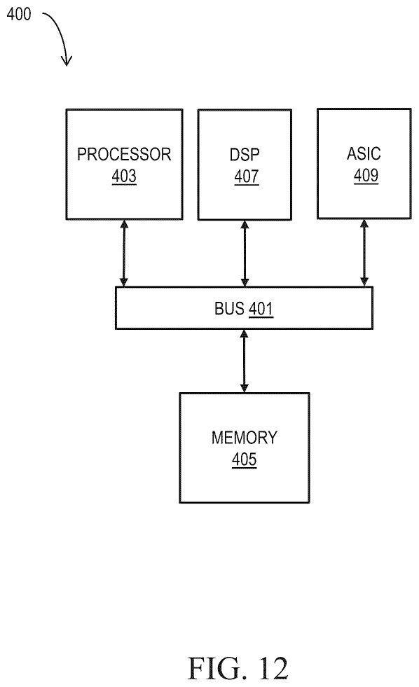

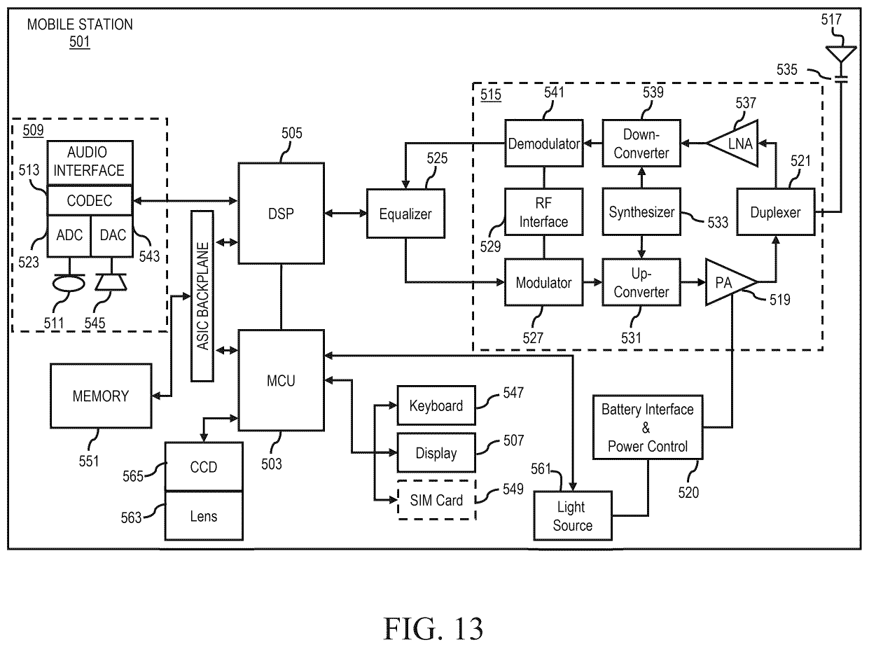

As shown in FIG. 4A, the controller 50 may be provided with a memory 51 for storing data thereon (e.g., sensor data from the sensor 46, the awareness pattern, etc.) and an awareness module 53 that may include a set of one or more instructions that cause the controller 50 to perform one or more steps of a method, such as the method 200 of the flowchart depicted in FIG. 8. In some embodiments, the processor or controller 50 may be a computer system as described below with reference to FIG. 11, a chip set described below with reference to FIG. 12 or a mobile terminal described below with reference to FIG. 13. In an example, the controller 50 may comprise any suitable type of mechanism that may cause the volatile composition dispenser to change the volatile composition being emitted. In the embodiment shown, the controller 50 may control the activation of the evaporative assistance elements, such as heaters, so that the heater will be turned on for the volatile composition that is desired to be emitted. Suitable controllers 50 may include, but are not limited to, analog timing circuitry, digital circuitry, combinations of analog and digital circuitry, microprocessors, and mechanical actuation switches such as shape memory alloys (NiTi wire) or bimetallic switches.

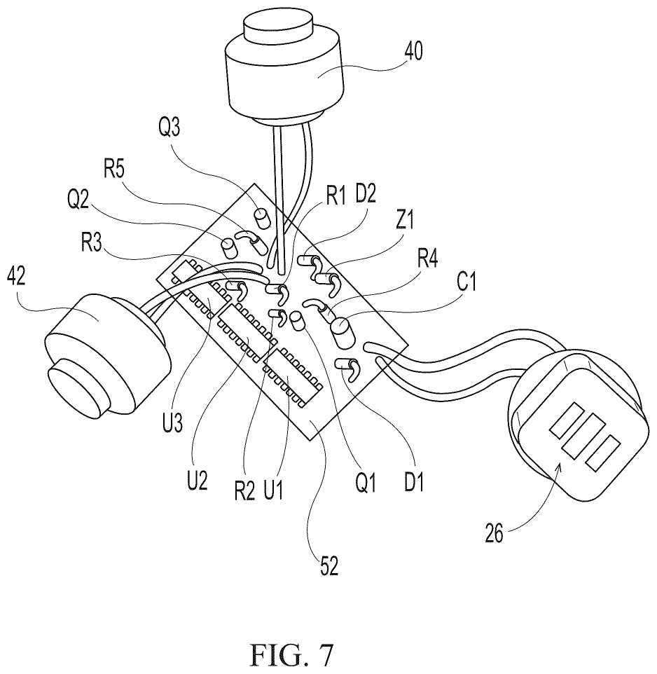

With reference to FIGS. 2 and 7, the controller 50 may comprise a combination analog and digital circuit in the form of a printed circuit board (or “PCB”). The circuit may include, for in a non-limiting example: a single-sided PC board 52; a capacitor designated C1; a pair of diodes D1 and D2; three transistors Q1, Q2, and Q3; five resistors R1-R5; three counters U1, U2, and U3; a third diode Z1. Where the evaporative assistance elements are heaters, any suitable type of heater may be used including, but not limited to, resistance heaters (several types of which are commercially available). The heaters 40 and 42, as well as the power source 26, shown as a wall-mount power plug in FIG. 7, may also be connected to the circuit board 52 by wires 66. Suitable components for circuit are set out in the following table.

TABLE 1

| Reference | | |
|---|---|---|
| Number or Letter | Component | Properties |
| C1 | Capacitor, Electrolytic | 1 microF, 250 V |
| D1, D2 | Diode | 1N4004, or similar |
| 26 | Wall power plug | |
| Q1, Q2, Q3 | Transistors, NPN | NPN 200 V, 200 mA |
| R1-R5 | Resistors | ⅛ watt |
| U1, U2, U3 | Counters | CD4024, or similar |
| Z1 | Diode, Zener, 11 V | 1N4741A, or similar |

The components of the circuit may be through-hole or surface mounted. In the configuration shown in FIG. 7, a 38×66 mm single sided PC board 52 with through-hole components is used. The material comprising the PC board 52 may be a standard material such as FR-4 epoxy base fiberglass, but any UL approved material is acceptable. The power source 26 shown in FIG. 7 may be a molded wall plug with approximately 100 mm pigtails into the PC board.

The controller 50 may include, but is not limited to, the following alternative types of controllers: (1) a magnetic sensor with a pickup that counts the number of rotations of the motor of a fan used to disperse the volatile composition(s) such that after a certain number of rotations, the volatile composition dispenser will switch from one volatile composition to another; and (2) a volatile composition dispenser comprising dual shape memory alloys, or bimetallic strips or switches that may complete a circuit at ambient temperature and then cut-off when a certain temperature is reached. The two-way effect may be used since as the temperature lowers, the material may complete the circuit again, thus acting as a thermostat to keep the heater on and then turn it off. The shape memory alloy may serve as the heater as well as the pulse generator.

The volatile composition dispenser 20 may comprise a number of additional optional features. The volatile composition dispenser may be provided with indicators so that a person is further made aware that the volatile material being emitted has changed, such as when the dispenser 20 is operating in the automatic mode (e.g., during predicted future low awareness periods) or in the manual mode (e.g., instances other than predicted future low awareness periods). Such indicators may be visual and/or audible, such as lights or sounds, respectively. For example, in the case of scented materials, such an indicator may allow a person to see which scent is being emitted at a given time. With reference to FIG. 3, the indicators may be in the form of lights 70, 72. In another example, at least a portion of the volatile composition dispenser 20 (such as all or a portion of the housing) or the reservoirs may be made of a type of plastic that changes color when heated.

The volatile composition dispenser may be provided with additional user controls. The volatile composition dispenser may include the user interface 55 which may include a power switch to allow a user to turn the volatile composition dispenser ON and OFF without removing it from the electrical socket. The volatile composition dispenser may be provided with a control that allows the user to control the discrete emission period of one or more of the volatile compositions, and/or the time between the emission of the different volatile compositions, or the time that the volatile materials are emitted during a simultaneous operation period. For example, in one non-limiting example, if the volatile composition dispenser is provided with the capability of emitting each volatile material during a period greater than 15 minutes and less than or equal to 48 hours, then the volatile composition dispenser may be provided with a control that allows the user to set the discrete emission period for one or more of the volatile compositions to 30 minutes, 45 minutes, or 72 minutes, or to one hour, for example.

The volatile composition dispenser may be provided with additional user controls. The volatile composition dispenser may comprise a thermostat or other switch in the user interface 55 to allow a user to adjust the temperature settings of the heat sources for one or more of the volatile compositions. As previously discussed, in one example the user interface 55 may feature a switch to adjust a desired heating temperature of the delivery engine 38 (e.g., wick) in one or both of the manual mode or automatic mode. The settings may be predefined for particular volatile compositions or may be adjustable based on selected temperatures to be applied to a wick. The settings of the user interface 55 may include a LOW and HIGH settings or LOW, MEDIUM, and HIGH settings, for example, that a user may set either directly on the volatile composition dispenser or remotely through a remote control (computer, phone, etc.). A device may have one, two, three, four, five, six, or more different intensity settings. The settings may be labeled as an intensity (i.e., HIGH, MEDIUM, LOW, etc.) or room-type (i.e., bathroom, bedroom, living, kitchen, etc.).

The volatile composition dispenser may also include the sensor(s) 46 and the volatile composition dispenser may be programmed to adjust for the readings of the sensors. For example, although FIG. 4A depicts a single sensor 46 in other examples more than one sensor 46 may be employed to provide sensor data to the controller 50. In one example, the volatile composition dispenser 20 may include sensors 46 such as temperature sensors, relative humidity sensors, volatile material sensors, light sensors (e.g., detecting day/night), and the like.

The volatile composition dispenser may be communicably connectable with various components of the dispenser, including the sensor(s) 46, evaporative assistance elements 40, 42, user interface 55, etc., using a wireless communication link. Various wireless communication links may be used, including 802.11 (Wi-Fi), 802.15.4 (ZigBee, 6LoWPAN, Thread, JennetIP), Bluetooth, combinations thereof, and the like. Connection may be through an ad hoc Mesh Network protocol. The controller 50 may include a wireless communication module in order to establish a wireless communication link with the controller 50 with various components of the system. Any module known in the art for establishing such communication links may be utilized. The controller 50 may include utilize a machine learning algorithm, such as a NEST® learning thermostat.

The cartridge 76 or reservoir 28, 30 may include an identification tag, such as an RFID tag and the housing 22 of the volatile composition dispenser 20 may include an RFID tag reader. An RFID tag may be used to tell the controller 50 details about the volatile composition contained in the cartridge 76 or reservoir 28, 30, such as the scent. The volatile composition dispenser 20 may include programs that adjust to account for information read from the RFID tag.

The volatile composition dispenser may include a tactile switch or registration point that, upon coming in contact with a cartridge or reservoir, provides signals to the volatile composition dispenser including, but not limited to, a new or refilled cartridge or reservoir that is full of a volatile composition has been inserted, an old cartridge or reservoir has been removed, etc. The PCB may interpret these signals and cause the volatile composition dispenser to act to programmed instructions accordingly, such as starting the total emission program for a new or refilled cartridge that is “full” of a volatile composition.

The volatile composition dispenser may also be sold in the form of a kit that includes the volatile composition dispenser 20 and one or more reservoirs 28, 30 of volatile compositions. The volatile composition dispenser 20 and/or kit may also include instructions for use that instruct the user regarding certain discrete emission periods that may be used to produce certain results, and/or instructions regarding where to place the volatile composition dispenser in a given space. For example, the instructions may include instructions for setting the volatile composition dispenser based on the size of the room, vehicle, etc. in which the volatile composition dispenser is placed. Such instructions may also include instructions to the user to choose more frequent changes between the emissions of scented materials for greater scent awareness. Instructions may also be provided to specify how to operate the volatile composition dispenser relative to other volatile composition dispensers. The instructions may be provided in any suitable form, e.g., written, audio, and/or video. Additional instructions may be provided, including where to position the dispenser 20 within an area (e.g., room of a house) such that the sensor 46 is positioned to capture an adequate amount of sensor data (e.g., light data, motion data, etc.) in order for the controller 50 to determine the awareness pattern of the area, as discussed in the method herein. For example, the instructions may advise not to position a light sensor 46 in a region where little to no light would be incident on the light sensor 46 over an extended time period (e.g., 24 hours). Similarly, for example, the instructions may advise not to position a motion sensor 46 in a region where little to no motion would be detected by the motion sensor 46 over an extended time period (e.g., 24 hours).

The volatile composition dispenser 20 may include a power source 26, such as a plug or battery. The volatile composition dispenser 20 may be battery powered so that it need not be plugged into an electrical outlet 24. If a plug is used as the power source to connect to an electrical outlet 24, the plug may include a cord or may be a wall-mount plug. The volatile composition dispenser 20 may also be configured so that it may be both plugged in and powered by a source of electrical current, and also battery powered. The volatile composition dispenser 20 may also be provided with an adapter so that it may be plugged into the cigarette lighter in a vehicle. In addition, the volatile composition dispenser 20 may be provided with a remote control that allows the user to control any, or all, of the emission properties of the volatile composition dispenser 20 (e.g., changing the volatile material being emitted) without touching the volatile composition dispenser.

The volatile composition dispenser 20 may comprise the controller 50 which may be a microprocessor that has less component parts compared to analog circuits, and improved circuit quality from lot to lot. The microprocessor may allow the user to program and control the temperature profile by modulation to alter performance. If desired, the microprocessor may be connected to the user interface 55. This may be any suitable type of user interface. Examples of types of user interfaces 55 include, but are not limited to, LCD screens and LEDs, buttons (push buttons or buttons that move side-to-side), dials, and the like. In addition, the microprocessor enables components to allow multiple volatile composition dispensers (such as those located in different parts of a room, or in different rooms), to communicate with each other. For example, the microprocessor may enable a remote control to send digital signals via an infrared beam to turn another volatile composition dispenser ON or OFF.

The evaporative assistance elements 40, 42, 44, such as a heater or fan, may be programmed to operate in various operational conditions. As will be discussed in more detail below, the evaporative assistance elements 40, 42, 44 may be configured to operate in an automatic mode (e.g., during a low awareness period) and operate in a manual mode (e.g., during times other than a low awareness period). During the automatic mode, the evaporative assistance elements 40, 42, 44 may operate at a lower setting (e.g., lower temperature for the heaters, lower speed for the fan) in order to reduce and/or minimize the rate of volatilization during periods of no or minimal subject awareness. Similarly, during the manual mode, the evaporative assistance elements 40, 42, 44 may operate at a higher setting (e.g., higher temperature for the heaters, higher speed for the fan) in order to increase the rate of volatilization during periods other than no or minimal subject awareness. As previously discussed, the evaporative assistance elements 40, 42, 44 may operate in the manual mode upon receiving a first signal from the controller 50 and may operate in the automatic mode upon receiving a second signal from the controller 50.

### Method For Operating a Device

A method is now discussed for operating the volatile composition dispenser. The method may involve capturing sensor data of an area (e.g., room in a home) from the sensor 46 and using the sensor data to determine an awareness pattern of the area. The awareness pattern may include one or more low awareness periods where a subject has no or minimal level of awareness of the area and one or more high awareness periods where the subject has a high level of awareness of the area. The method may utilize the awareness pattern to make operational decisions regarding the volatile composition dispenser (e.g., when to put the dispenser in the manual mode and when to switch the dispenser to the automatic mode). For example, the method may utilize the awareness pattern captured over a first time period to predict one or more future low awareness periods over a second time period after the first time period. The method may then make operational decisions (e.g., switching the volatile composition dispenser to the manual mode or automatic mode) depending on these one or more future low awareness periods.

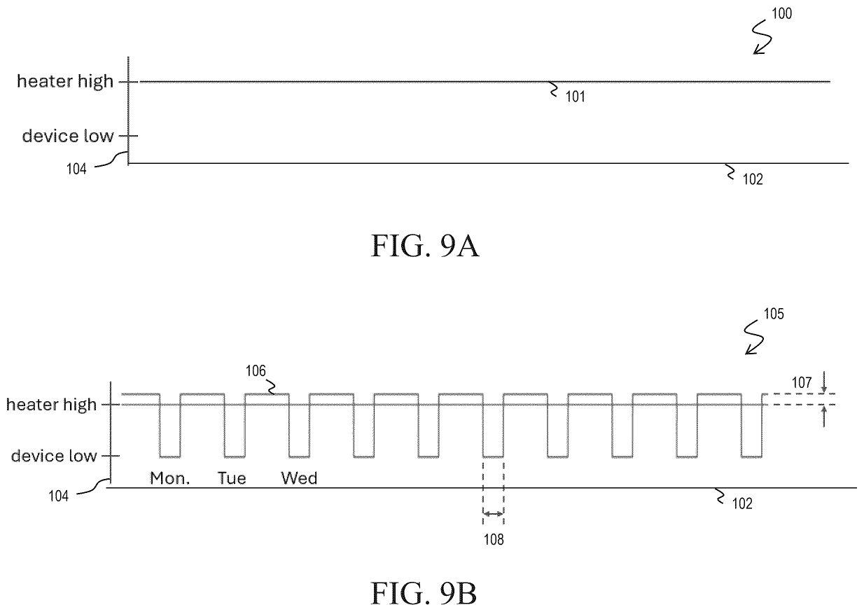

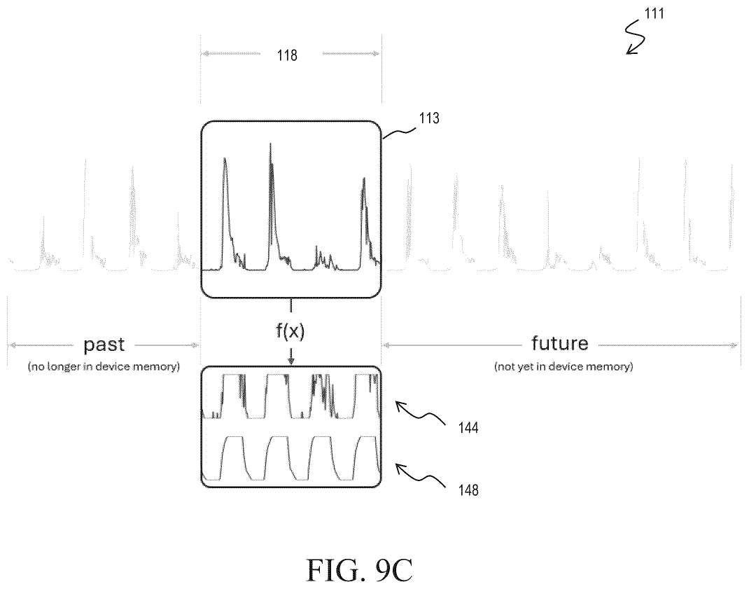

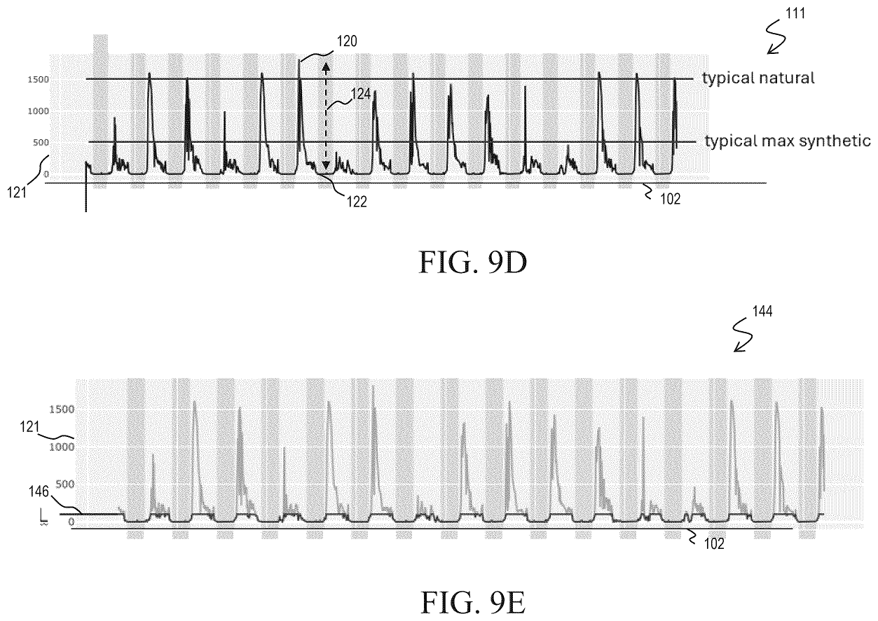

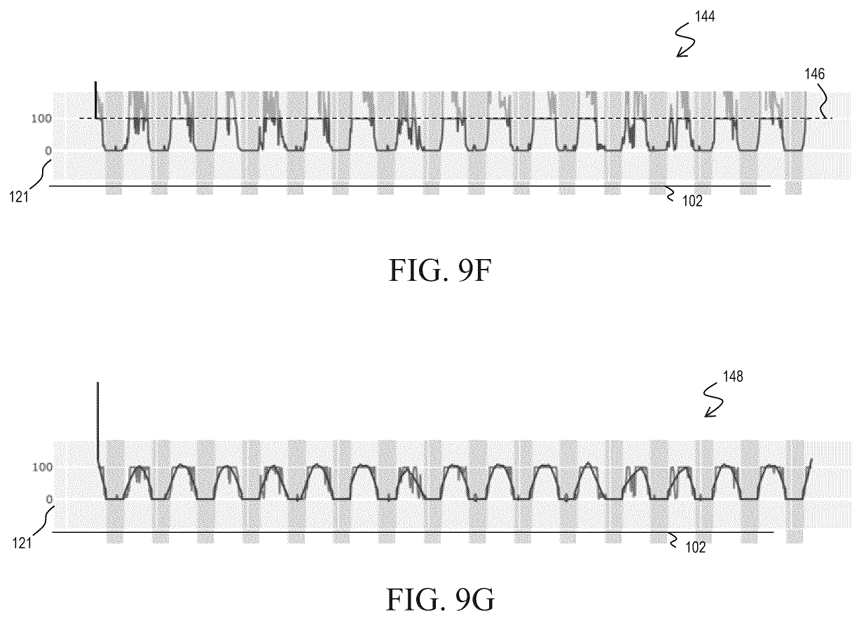

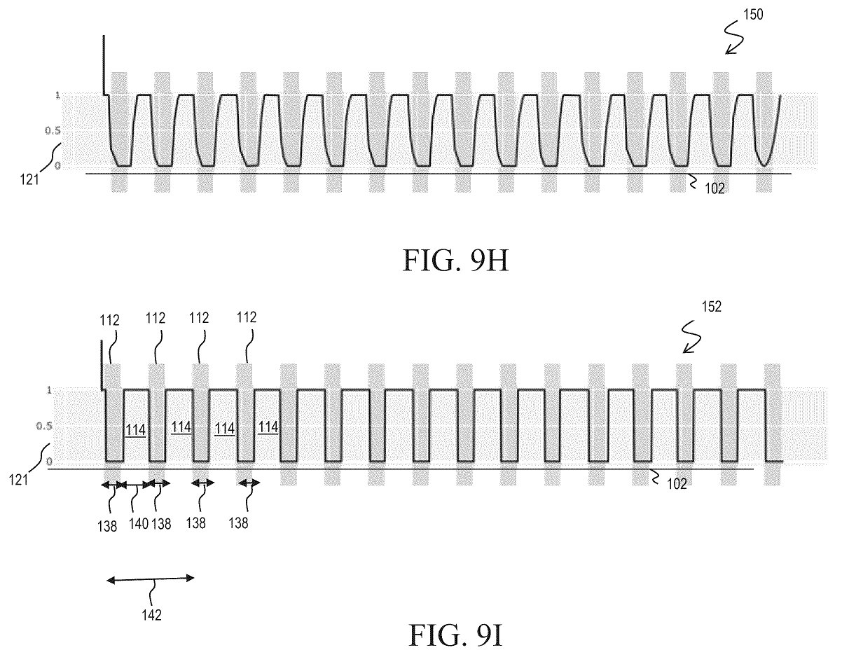

FIG. 9A depicts an example of a signal transmitted from the controller to the evaporative assistance element based on a conventional method of operating a conventional volatile composition dispenser. The horizontal axis 102 is time in arbitrary units. The vertical axis 104 is temperature in arbitrary units. As depicted in FIG. 9A, a graph 100 is shown with a signal 101 that may be transmitted from the controller to the evaporative assistance element (e.g., heater) over time in a conventional volatile composition dispenser. In an example, this signal 101 may be indicative of a conventional volatile composition dispenser operating in a constant manual mode, where the delivery engine is heated by the evaporative assistance element (e.g., heater) to a fixed temperature. It was recognized that this signal 101 transmitted in a conventional volatile composition dispenser has drawbacks, including dispensing the volatile composition during time periods with no or minimal subject awareness of the area.

FIG. 9B depicts a graph 105 with a signal 106 that may be transmitted from the controller 50 to the evaporative assistance element 40, 42 (e.g., heater) based on the improved method and improved volatile composition dispenser disclosed herein. Unlike the signal 101 of the conventional volatile composition dispenser of FIG. 9A, the signal 106 of FIG. 9B may generally follow a pattern (e.g., square wave pattern), where the signal 106 may go from a low value during periods 108 of low subject awareness of the area to a high value during periods of high subject awareness of the area. Consequently, the signal 106 based on the method disclosed herein may minimize or eliminate the dispensation of the volatile composition during periods 108 of low subject awareness. Additionally, as shown in FIG. 9B, the signal 106 may have a maximum value (e.g., temperature) that exceeds the maximum value of the conventional signal 101 by an incremental temperature value 107. Thus, not only does the signal 106 minimize dispensing of the volatile composition during low awareness periods, it may also heat the evaporative assistance elements 40, 42 (e.g., heater) to a higher temperature during high awareness periods and thus enhances the noticeability of the volatile composition during periods of high subject awareness.

A flowchart that shows one or more steps of the method will now be discussed herein. FIG. 8 is a flow chart that illustrates an example of a method 200 for operating a device, such as the volatile composition dispenser 20. Although the flow diagram of FIG. 8 may be depicted as integral steps in a particular order for purposes of illustration, in other embodiments one or more steps, or portions thereof, are performed in a different order, or overlapping in time, in series or in parallel, or are deleted, or one or more other steps may be added, or the method may be changed in some combination of ways.

In one example, the volatile composition dispenser 20 may be selected from the group comprising an air freshening device, an air treatment device and a pesticide dispensing device.

An initial step of the method 200 may now be discussed, that may involve capturing first data or sensor data of the area (e.g., room in a house) with a sensor. In step 202, the sensor 46 may capture sensor data of the area 10 and may transmit the sensor data to the controller 50. In one example, the sensor 46 may capture sensor data at regular time increments (e.g., every 15 minutes) over a first time period and may transmit the sensor data to the controller 50. In step 202, this sensor data may be received by the controller 50. As previously discussed, the sensor 46 may be one or more of a light sensor, a motion sensor, an image sensor (e.g., camera), a location sensor (e.g., GPS sensor in a smartphone) and/or a sensor to indicate activity of one or more devices on a network (e.g., Wi-fi network in a home).

In one example, step 202 may be performed over the first time period which may be in a range from about 10 hours to about 10 days and/or from about 12 hours to about 6 days and/or from about 18 hours to about 3 days. FIG. 9C depicts that the sensor data captured in step 202 may be performed over a time window 113 that has a duration that is the first time period 118. The sensor data 111 depicted in FIG. 9C that may be captured over the time window 113 may be stored in the memory 51 of the controller 50. As shown in FIG. 9C, the time window 113 may shift over time such that sensor data from the sensor 46 may be continuously captured over the moving time window 113 and stored in the memory 51 of the controller 50 having a duration corresponding to the first time period 118. The duration of the first time period 118 may be sufficient for the method 200 to determine an awareness pattern of the area that includes one or more low awareness periods and one or more high awareness periods. In one example, where light sensor data may be utilized to determine the awareness pattern based on a circadian pattern, the first time period may be at least 18 hours to 24 hours, in order to capture at least one low awareness period (e.g., night period or dark period where an amount of light may be below a threshold value) and at least one high awareness period (e.g., day period or light period where the light sensor data may indicate an amount of light above a threshold value).

A subsequent step of the method 200 may now be discussed, where the controller 50 may process the sensor data from the sensor 46 that was received in step 202. In step 204, the controller 50 may determine second data, based on the sensor data from step 202, where the second data may indicate an awareness pattern of the area over the first time period. For example, as shown in FIG. 9D, the sensor data 111 that may be received from the sensor 46 in step 202 over time is shown in a graph. The horizontal axis 102 is time in arbitrary units (e.g., extends about 1 to 2 weeks). The vertical axis 121 is light data in units of lux. As shown in FIG. 9D, the sensor data 111 may indicate that the light sensor data from step 202 has a pattern including one or more maximum values 120 with one or more minimum values 122 in between consecutive maximum values 120. These maximum values 120 may correspond with light periods (e.g., day period) where the light sensor data 111 may exceed a light threshold and the minimum values 122 may correspond with dark periods (e.g., night period) where the light sensor data 111 may be less than the light threshold. In an example, the light threshold may be in a range from about 0 lux to about 10 lux and/or in a range from about 0 lux to about 20 lux. In an example, FIG. 9D depicts a difference 124 between the maximum value 120 and the minimum value 122, such as over a portion (e.g., one day) of the first time period 118.

In step 204, in addition to determining the plot of FIG. 9D showing the sensor data 111 over time, the controller 50 may process the sensor data 111 of FIG. 9D. In one example, as shown in FIGS. 9E and 9F, in step 204 the controller 50 may suppress sensor data 111 above a certain threshold 146 (e.g., about 100 lux and/or in a range from about 3 lux to about 4000 lux and/or in a range from about 2 lux to about 500 lux and/or in a range from about 1 lux to about 200 lux). As shown in FIGS. 9E and 9F the suppressed data 144 may be depicted which may show the sensor data 111 of FIG. 9D, where the sensor data above the threshold 146 may be suppressed (and set equal to the threshold 146, such as about 100 lux in one example). In an example, this suppression of the sensor data may involve clipping (or throwing away) data collected above the threshold 146 (e.g., 100 lux) and thus only use the data between 0 and the threshold 146 value (e.g., between 0 lux and 100 lux). Synthetic light total intensity may be typically far lower than natural light sources, and this limited range may still provide a clear signal of a state for both synthetic and natural light sources. In an example, eliminating the sensor data above the threshold 146 (e.g., data >100 lux) may also provide the benefit that a high degree of response variation (noise) is eliminated, due to changing conditions which may cause minor fluctuations in signal amplitude (e.g., clouds, momentary obscuration of sensor, etc.)

In step 204, in addition to performing the suppression of the sensor data 111 above the threshold 146, the controller 50 may process the suppressed data 144 by smoothing the data to obtain a smoothed curve 148, as depicted in FIG. 9G. The smoothing operation performed by the controller 50 in step 204 may be any smoothing operation appreciated by one of ordinary skill in the art. In an example, performing the smoothing operation to the data may advantageously further eliminate noise and allow the fundamental circadian pattern in the sensor data to be easily observed. However, in some examples, the smoothing operation may also introduce error into the low awareness periods (dark periods), modifying from a continuous zero lux sequence to having some false-positive light response. Thus, in an example, in the method the smoothing operation may first be performed followed by replacing smoothed low awareness periods (smoothed dark periods) with zero wherever the sensor data was originally near zero.

In still another example, in step 204 the controller 50 may process the smoothed curve 148 by normalizing the values, in order to obtain normalized data 150 (e.g., where the range of values on the vertical axis 121 are normalized between 0 and 1). As appreciated by one skilled in the art, the normalization of step 204 may be performed by rescaling the values of the smoothed curve 148. For example, a maximum value of the smoothed curve 148 may be set to 1, a minimum value of the smoothed curve 148 may be set to 0 and the other values of the smoothed curve 148 may be set to respective values between 0 and 1 based on consistent scaling. In other examples, the normalization of the smoothed curve 148 may be performed using any known technique appreciated by one skilled in the art. In some examples, the normalizing may be performed using a rolling delta function. As appreciated by one skilled in the art, this function may calculate delta values over a rolling window using a Savitzky-Golay or moving average filter. It was recognized that converting the smoothed curve 148 into normalized data 150 may advantageously enable the algorithm of the method 200 to handle data from a great number of light sensor response specifications. The normalization is described relative to the smoothed curve 148, but the sensor data 111 or the suppressed data 144 may be normalized without a prior smoothing operation.

In yet another example, in step 204 the controller 50 may process the normalized data 150 to obtain digitized data 152 where the values of the digitized data 152 are either 0 or 1 over the first time period. As appreciated by one skilled in the art, the digitization of the data 152 in step 204 may be performed by assigning the values of the normalized data 150 below a threshold value (e.g., 0.5) to have a value of 0 and by assigning all values of the normalized data 150 above the threshold value to have a value of 1. In other examples, the digitization of the normalized data 150 may be performed using any known technique appreciated by one skilled in the art. In an example, the conversion of the normalized data 150 to the digitized data 152 may advantageously ease of further calculations. For example, prior to the conversion to the digitized data 152, a small amount of the smoothing may be applied to ensure artifacting (e.g., which may have survived the previous filters) and thus does may not distort the digital transform. In another example, a centered average may be applied to prevent shifting at entry and exit points of the low awareness periods. In some examples, a criteria for conversion of the normalized data 150 to digitized data 152 may include hyperparameters which are optimized against a reference labeled dataset. The digitization is described relative to the normalized data 150, but the sensor data 111 or the suppressed data 144 may be digitized without a prior normalizing operation and/or without a prior smoothing operation.

In one example, the digitized data 152 depicted in FIG. 9I may be an awareness pattern 110 that may include a plurality of low awareness periods 112 (e.g., 0 value of the digitized data 152, indicative of no or a low level of subject awareness of the area) and a plurality of high awareness periods 114 (e.g., 1 value of the digitized data 152, indicative of a high level of subject awareness of the area). For ease of illustration, only four low awareness periods 112 and four high awareness periods 114 are labelled in FIG. 9I. As shown in FIG. 9I, in one example, a duration 138 of the low awareness periods 112 and a duration 140 of the high awareness periods 114 is also depicted and labeled.

The method 200 may next involve step 206 that may include determining a value of a characteristic of the sensor data (from step 202) or a characteristic of the awareness pattern (from step 204). The characteristics may be used as an indicator of the reliability that the sensor data (from step 202) and/or the awareness pattern (from step 204) to accurately reflect the level of subject awareness in the area and thus to provide a reliable basis to make operational decisions regarding the volatile composition dispenser 20. Thus, the method 200 in subsequent steps may make an operational decision of the volatile composition dispenser 20 (e.g., manual mode or automatic mode) based on the values of these characteristics, if each of these characteristics meet certain criteria.

In step 206, a first characteristic may be determined of the sensor data from step 202. In one example, this first characteristic may be a level of continuity of the sensor data. As previously discussed, in step 202 the sensor data from the sensor 46 may be measured at regular time increments (e.g., every 15 minutes) and received at the controller 50. Thus, in one example, the level of continuity of the sensor data determined in step 206 may be based on whether sensor data was not received by the controller 50 in step 202 at one or more regular time increments. In one example, the level of continuity may indicate a number of regular time increments when sensor data was not received by the controller 50 and/or a ratio of the number of regular time increments when sensor data was received to a total number of regular time increments in the first time period. For example, the sensor data 111 in FIG. 9D may indicate that sensor data was received at every regular time increment over the first time period 118 and thus in that example the number of regular time increments where no sensor data was received is 0 and thus the level of continuity is high. In another example, where the sensor data is measured at 15 minute regular time increment over 60 total time increments (15 hour time period), the level of continuity is based on a ratio of a number of regular time increments (e.g., 58) when sensor data was received to the 60 total time increments or 0.97.

In step 206, a second characteristic may be determined of the sensor data received in step 202. In this example, the second characteristic may be the difference 124 (FIG. 9D) between the maximum value 120 and the minimum value 122 of the sensor data 111 over the first time period. In one example, the difference 124 may be calculated using a function which computes the range (e.g., maximum value 120-minimum value 122) over a specified window size. While the algorithm of the method may see light to dark transitions with even a very low amount of available light, there may be a point at which accuracy will break down due to various sources of noise. Thus, by determining the difference 124 or a total range (lightest to darkest) of the light data 111 within a window of time, the method may assess if the available range is significant enough to proceed with further calculation. In some examples, in step 208 the difference 124 may be compared with a predetermined value range in order to decide whether the sensor data 111 is sufficiently reliable to make future predictions regarding a future low awareness period.

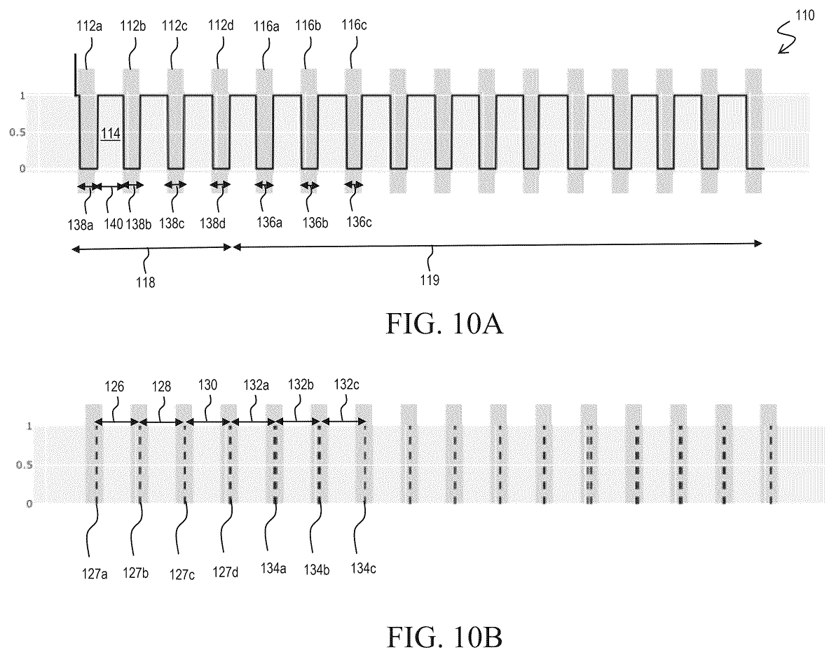

In step 206, a third characteristic may be determined of the awareness pattern. FIG. 10A depicts the awareness pattern 110 that may include a plurality of low awareness periods 112a, 112b, 112c, 112d over the first time period 118. FIG. 10B depicts centers 127a, 127b, 127c, 127d of the respective low awareness periods 112a, 112b, 112c, 112d over the first time period 118. FIG. 10B further depicts a first distance 126 between the centers 127a, 127b of a first consecutive pair of low awareness periods 112a, 112b, a second distance 128 between the centers 127b, 127c of a second consecutive pair of low awareness periods 112b, 112c and a third distance 130 between the centers 127c, 127d of a third consecutive pair of low awareness periods 112c, 112d. In one example the third characteristic may be a difference between the first distance 126 and the second distance 128 or a difference between the second distance 128 and the third distance 130. In another example, the third characteristic may be a standard deviation between two or more of the first distance 126, second distance 128 and/or third distance 130. Although three distances 126, 128, 130 are depicted in FIG. 10B, this is merely one example of an awareness pattern and the third characteristic in step 206 may be determined based on less or more than two distances between three different consecutive pairs of low awareness periods. In some examples, the standard deviation of the distances 126, 128, 130 may be calculated between the centers of the low awareness periods 112a, 112b, 112c, 112d, using a rolling window. In an example, the standard deviation of these distances 126, 128, 130 may be used to assess if the regularity of the dark/light periods (low awareness periods/high awareness periods) is consistent enough to enable accurate prediction of future dark periods (predicted future low awareness periods). In step 208, the resulting value may be compared against an empirically selected threshold max. In still other examples, the third characteristic may be a difference between a first distance between a first pair of consecutive high awareness periods 114 and a second distance between a second pair of consecutive high awareness periods 114. In this example, this difference may be calculated in a similar manner as the difference between the first distance 126 and the second distance 128 for the low awareness periods 112 (e.g., using a standard deviation).

In step 206, a fourth characteristic may be determined of the awareness pattern. FIG. 9I depicts that the low awareness periods 112 of the awareness pattern 110 have a duration 138 and the high awareness periods 114 of the awareness pattern have a duration 140. In one example, the fourth characteristic may be a value of one or more of the durations 138 of the low awareness periods 112. In another example, the fourth characteristic may be a value of one or more of the durations 140 of the high awareness periods 114.

In step 206, a fifth characteristic may be determined of the awareness pattern. In one example, the fifth characteristic may be a number of the one or more low awareness periods 112 and/or the number of the one or more high awareness periods 114 within a fixed time interval. As shown in FIG. 9I, in one example the fixed time interval 142 is depicted that may have a value (e.g., about 24 hours and/or in a range from about 12 hours to about 36 hours and/or in a range from about 6 hours to about 48 hours). As shown in FIG. 9I, in this specific example the fifth characteristic may be two, since two low awareness periods 112 occur within the fixed time interval 142 or since two high awareness periods 114 occur within the fixed time interval 142. The number of low awareness periods 112 and high awareness periods 114 with the fixed time interval indicates the number of transitions between low awareness periods 112 and high awareness periods 114. There may be a minimum threshold of transitions in the fixed time period, such as greater than 1, or greater than 2.

Although step 206 may involve determining a value of one or more of the above discussed five characteristics, the characteristic whose value may be determined in step 206 is not limited to these specific five characteristics. In another example, the characteristic whose value is determined in step 206 may include any other characteristic of the first data (from step 202) or the awareness pattern (from step 204) whose value may tend to indicate a reliability of the awareness pattern to accurately predict one or more future low awareness periods in the area.

The value of the characteristic determined in step 206 may then be compared with a predetermined value range in step 208. As shown in FIG. 8, the method 200 may include a decision block 208 where the value of the one or more characteristics determined in step 206 may be compared with one or more respective predetermined value ranges for each respective characteristic.

For example, the predetermined value range for the first characteristic (level of continuity of the sensor data) may include a range of values for a maximum number of regular time increments where no sensor data was received over the first time period, such as between 0 and 10 or a range of values for a ratio of the number of regular time increments where sensor data was received to the total number of regular time increments, such as between about 0.5 and about 1.0 and/or between about 0.9 and about 1.0.

In another example, the predetermined value range for the second characteristic (difference between the maximum and minimum values of the sensor data) may be a range with values that ensure sufficient separation between a sensor reading indicative of darkness and a sensor reading indicative of light. In one example, this predetermined value range may be about 3 lux or greater. In yet another example, the predetermined value range may be between about 3 lux and about 200 lux. In yet another example, the predetermined value range may be between about 3 lux and about 100 lux. In yet another example, the predetermined value range may be between about 3 lux and about 50 lux. In yet another example, the predetermined value range may be between about 3 lux and about 10 lux.

In another example, the predetermined value range for the third characteristic (difference or standard deviation between the centers of consecutive pairs of low awareness periods) may be a range from about 0 minutes to about 400 minutes and/or a range from about 0 minutes to about 200 minutes.

In another example, the predetermined value range for the fourth characteristic (duration of the low awareness periods and/or duration of the high awareness periods) may be a range from about 3 hours to about 18 hours and/or within a range from about 4 hours to about 12 hours. In an example, the fourth characteristic value may indicate whether the duration 138 of each low awareness period 112 is within a specific range (e.g., between 3 and 18 hours). In an example, low awareness periods 112 which exceed a maximum threshold and may be less than a minimum threshold may be rejected as out of compliance with a typical circadian rhythm. In an example, low awareness periods 112 which are in compliance may then be classified as either shorter than or longer than an amount of time for which it is intended to operate the volatile composition dispenser 20 in the automatic mode. This may enable the method 200 to be selective in how the method 200 may initiate the automatic mode for a predicted future low awareness period.

In another example, the predetermined value range for the fifth characteristic (number of the low awareness periods 112 and/or high awareness periods 114 in a fixed time interval 142) may be a range from about 1 to about 10 and/or in a range from about 1 to about 3. In an example, if too many or too few low awareness periods and high awareness periods are observed in the fixed time interval 142, the data may be classified as out of compliance with a normal circadian pattern.

In decision block 208, a decision may be made as to whether a value of the characteristic determined in step 206 is within the respective predetermined value range for that characteristic. If the outcome of this decision is in the affirmative, the method 200 may move to decision block 210. If the outcome of this decision is in the negative, the method 200 may move to step 212.

In decision block 210, a determination may be made as to whether an additional characteristic of the sensor data or awareness pattern needs to be considered. If this determination is in the affirmative, the method 200 may go back to step 206 and the value of this additional characteristic may be determined followed by decision block 208 where that value of the additional characteristic may be compared with the predetermined value range for that additional characteristic. If the determination in the decision block 210 is in the negative, this means that there may be no additional characteristics of the sensor data or awareness pattern that need to be considered. The method 200 then moves to step 214. In some examples, only one of the above discussed characteristics are considered in steps 206 through 210. In other examples, two of the above discussed characteristics may be considered in steps 206 through 210. In other examples, three of the above discussed characteristics may be considered in steps 206 through 210. In other examples, four of the above discussed characteristics may be considered in steps 206 through 210. In still other examples, each of the five above discussed characteristics may be considered in steps 206 through 210. In still other examples, none of the above discussed characteristics but other characteristics of the sensor data or awareness pattern may be considered in steps 206 through 210.

In step 212, after a negative determination in decision block 208, this may indicate that the value of at least one characteristic determined in step 206 is not within the predetermined value range for that characteristic. Thus, the sensor data and/or awareness pattern may not be sufficiently reliable to accurately predict a future awareness period of a subject in the area, such as to predict a future low awareness period. Thus, in step 212 the method 200 may involve a step of operating the volatile composition dispenser in a manual mode (e.g., where the volatile composition is dispensed in accordance with a manual mode setting of the user using the user interface 55). In one example, in step 212 the controller 50 may transmit a first signal to the evaporative assistance element 40, 42 (e.g., heater) so to heat the delivery engine 38 (e.g., wick) in accordance with the temperature setting for manual mode based on the user interface 55 (e.g., low, medium, high, etc.).

In step 214, after a negative determination in decision block 210, this may indicate that the value of each characteristic determined in step 206 is within the predetermined value range for each characteristic. Thus, there may be no value of a characteristic of the sensor data or awareness pattern determined in step 206 that is outside the predetermined value range for the characteristic. Thus, the sensor data and/or awareness pattern may be sufficiently reliable to accurately predict a future awareness period of a subject in the area, such as to predict a future low awareness period. Thus, in step 214 the method 200 may first predict a future low awareness period of the awareness pattern over a second time period that is after the first time period.

As shown in FIG. 10A, in one example the awareness pattern 110 over the first time period 118 is depicted that may include four low awareness periods 112a, 112b, 112c, 112d. FIG. 10B further depicts the respective centers 127a, 127b, 127c, 127d as well as the first distance 126 between the centers 127a, 127b of the first consecutive pair of low awareness periods 112a, 112b; the second distance 128 between the centers 127b, 127c of the second consecutive pair of low awareness periods 112b, 112c; and the third distance 130 between the centers 127c, 127d of the third consecutive pair of low awareness periods 127c, 127d. In one example, in step 214 the controller 50 may determine a predicted future low awareness period 116a over a second time period 119 (after the first time period 118) by first determining a spacing 132a (FIG. 10B) between the center 127d of the last low awareness period 112d and a center 134a of the predicted future low awareness period 116a. In one example, the controller 50 may determine the spacing 132a based on averaging two or more of the first distance 126, the second distance 128 and the third distance 130. In another example, in step 214 the controller 50 may next determine a duration 136a of the predicted future low awareness period 116a. In one example, the controller 50 may determine the duration 136a based on averaging two or more of the durations 138a, 138b, 138c, 138d of the respective low awareness periods 112a, 112b, 112c, 112d. In one example the controller 50 may determine the predicted future low awareness period 116a based on having the duration 136a that is centered at the center 134a. The controller 50 may determine one or more other predicted future low awareness periods 116b, 116c, as shown in FIG. 10A using similar steps as previously discussed with respect to the determination of the predicted future low awareness period 116a. Although only three predicted future low awareness periods are labeled in FIGS. 10A and 10B, this is for ease of illustration.

In another example, in step 214 the controller 50 may determine a predicted future high awareness period over a second time period (after the first time period 118), using similar techniques employed in determining the predicted future low awareness period. For example, in step 214 the controller 50 may determine a spacing between a center of the last high awareness period and a center of the predicted future high awareness period. In this example, the controller 50 may determine this spacing based on averaging two or more of the first distance (between a first pair of consecutive high awareness periods), the second distance (between a second pair of consecutive high awareness periods) and the third distance (between a third pair of consecutive high awareness periods).

After predicting the future low awareness period in step 214, the method 200 may move to step 216 where the volatile composition dispenser 20 is operated in the automatic mode during the predicted low awareness period determined in step 214 (e.g., where the volatile composition is not dispensed or dispensed at a low temperature setting in accordance with an automatic mode setting of the user using the user interface 55). In one example, in step 216 during the predicted future low awareness period 116a the controller 50 may transmit the second signal to the evaporative assistance element 40, 42 (e.g., heater) so to not heat the delivery engine 38 (e.g., wick) or to heat the delivery engine 38 at a low temperature setting in accordance with the automatic mode based on the user interface 55 (e.g., low). This may advantageously reduce the dispensing of the volatized composition during the predicted future low awareness period, since there is little to no subject awareness of the area during this period.

In some examples, in step 216 the controller 50 may transmit the second signal to the evaporative assistance element 40, 42 such that the automatic mode is commenced at the center 134a of the predicted future low awareness period 116a. However, in other examples, the second signal may be transmitted such that the automatic mode is commenced at any time period within the predicted future low awareness period 116a. In still other examples, if the duration 136a of the predicted future low awareness period 116a is less than a low threshold value (e.g., about 3 hours and/or about 5 hours) then in step 216 the controller 50 may transmit the second signal to the evaporative assistance element 40, 42 prior to onset of the predicted future low awareness period 116a. This may be advantageous since the onset of a low awareness period may have an irregular start or end relative to the prediction average and the business logic may choose to bias the beginning or ending of the signal condition to provide noticeability at the leading or trailing edge of the awareness period as priority vs per-awareness period responsiveness. Further, business logic may choose to implement a standard signal duration to balance overall refill longevity duration as priority vs per-awareness period responsiveness.

In an example, the volatile composition dispenser 20 may have an automatic mode duration which may be a predetermined duration at which the controller 50 may transmit the second signal in step 216. In some examples, the transmission of the second signal in step 216 may be based on comparing the automatic mode duration with the duration 136a of the predicted future low awareness period 116a determined in step 214. Thus, in this example, in step 216 the controller 50 may determine when to transmit the second signal to the evaporative assistance element 40, 42 based on this comparison. For example, if the duration 136a is greater than the automatic mode duration (e.g., about 3 hours or in a range from about 2 hours to about 6 hours or in a range from about 1 hour to about 10 hours) then the controller 50 may transmit the second signal during an intermediate time (e.g., center 134a) of the predicted future low awareness period 116a. In another example, if the duration 136a is less than the automatic mode duration, then the controller 50 may transmit the second signal prior to onset of the predicted future low awareness period 116a. In still other examples, in step 216 the controller 50 may transmit the second signal for the entire duration 136a of the future low awareness period 116a or for a fraction of the duration 136a of the future low awareness period 116a.

In some examples, in step 216 prior to transmitting the second signal from the controller 50 to the evaporative assistance element 40, 42, the controller 50 may verify that the sensor data from the sensor 46 (e.g., light data) is below a low threshold (e.g., 10 lux) during the predicted future low awareness period 116a. This may advantageously verify that the predicted future low awareness period 116a is an actual low awareness period, prior to transmitting the second signal in step 216.

In another example, after predicting the future high awareness period in step 214, the method 200 may move to step 216 where the volatile composition dispenser 20 may be operated in a certain mode (e.g., manual mode) during the predicted high awareness period. In one example, in step 216 the volatile composition dispenser 20 may be operated so to evenly distribute a device operation during the predicted future high awareness period. In one example, in step 216 the controller 50 may transmit the second signal to the evaporative assistance element 40, 42 such that the manual mode may be commenced at the center of the predicted future high awareness period. However, in other examples, in step 216 the controller 50 may transmit the second signal to the evaporative assistance element 40, 42 so to schedule device activities with respect to the center of the predicted future high awareness period.

After step 216, the method 200 may move to decision block 218 where it is determined whether the user has inputted a request to terminate the method (e.g., with the user interface 55). If this decision is in the affirmative, the method 200 may end. If this decision is not in the affirmative, the method 200 may proceed back to step 202. As previously discussed, the sensor 46 may continuously capture sensor data during the method 200 and thus the time window 113 (FIG. 9C) may continuously move and store sensor data in the memory 51 of the controller 50 over a duration of the first time period 118. Thus, in this example, the awareness pattern of FIG. 10A may be continuously updated as the time window 113 moves, based on the repeating of steps 202 and 204 and the values of the characteristics may also be continuously updated, based on repeating step 206. For example, as steps 202 through 206 may be repeated as the time window 113 moves, what were the predicted future low awareness periods 116a, 116b, 116c (FIG. 10A) may become actual low awareness periods of the awareness pattern and in step 214 these actual low awareness periods may be used to predict still future low awareness periods in step 214, as the awareness pattern is updated.

Unlike conventional methods, the method disclosed herein may include an algorithm that performs a series of ongoing quality checks on the incoming light sensor data to determine whether and when to enter the “low mode” each day, based on steps 206 through 210. Thus, in an example, the determination in block 208 for each characteristic value may be a “quality check” and if each of those quality checks are in the affirmative (i.e., yes determination in step 208), then the method may proceed to predict a future low awareness period during which to activate the automatic mode. If the collected data fails any of the quality checks (based on a negative determination in any iteration of step 208), the volatile composition dispenser 20 may remain in the manual mode.

In an example, the method 200 may include an algorithm designed to protect a business model and claims by applying the automatic mode (e.g., a blanket low-mode duration) to all devices or volatile composition dispensers which meet the necessary criteria (e.g., steps 206 through 210) to be operated in the automatic mode. This may ensure that “fuel” (volatile composition) utilization rate is consistent and compatible with a refill duration for the cartridges 76 of the volatile composition dispenser. In another example, the method 200 disclosed herein may feature an algorithm designed to prevent low operation mode (automatic mode) from occurring during high awareness periods, and applying a middle-out approach (manual mode) during low awareness periods. To do this, the method 200 may first establish high confidence that a typical circadian pattern has been identified (e.g., based on satisfying the criteria of the characteristics in steps 206 through 210) and then proceed to predict (e.g., in step 214) when the next dark period (e.g., predicted future low awareness period) will occur. This may enable the method to initiate a low mode (e.g., automatic mode) around a mid-point of the dark period rather than at a trailing or leading edge of the dark period (e.g., predicted future low awareness period).

In an example, the iteration of steps 206 through 210 for each characteristic value may be viewed as cascading quality checks and as key elements of an algorithm to accurately predict an upcoming nighttime (e.g., predicted future low awareness period) and put the volatile composition dispenser 20 into the automatic mode. In an example, each characteristic or quality checks may involve some calculation, manipulation, and/or comparison (e.g., in steps 206 through 210) of the light data collected through the light sensor 46. In another example, the iteration of steps 206 through 210 for each characteristic may be viewed as a series of conditional trapdoors along a long walkway. If each characteristic falls within the respective predetermined value range in step 208, then the method 200 may not fall into any of the quality checks in the sequence and then and only then is automatic mode enabled by prediction of the next night time period (in steps 214 and 216).

Although the method 200 is discussed above with respect to the volatile composition dispenser 20 that operates in the automatic mode during a predicted future low awareness period and in the manual mode otherwise, for other volatile composition dispensers. The method may be modified, depending on the type of volatile composition, so that the volatile composition dispenser 20 operates in the automatic model during a predicted future high awareness period and in the manual mode otherwise. For example, where the volatile composition dispenser 20 is a pesticide dispenser, it may be advantageous to reduce or refrain from dispensing the pesticide in an area during a high awareness period and to dispense the pesticide at a regular rate otherwise (e.g., during low awareness period). This may be due to the advantageous outcome of minimizing human exposure to the dispensed pesticide. In this example, the method 200 may be modified such that step 214 may involve predicting a future high awareness period, using similar techniques are previously discussed herein with respect to predicting a future low awareness period. In step 216, the controller 50 may then switch the pesticide dispenser into the automatic mode during the predicted future high awareness period, since it would be advantageous to reduce or eliminate dispensation of the pesticide in the area during high awareness periods with a high level of subject awareness of the area.

### Results of Applying the Method

After developing the method disclosed herein, data may be collected to verify the rate of accuracy of the method 200 in prediction of the future low awareness period (or future high awareness period). Based on the collected data, employing the method disclosed herein with the quality checks of the characteristic values (i.e., steps 206 through 210) may have a very high rate of ensuring the volatile composition dispenser correctly enters into the automatic mode (low-energy mode) during regularly timed future predicted low awareness periods (e.g., dark/sleeping periods) in a household.

The collected data may include data regarding the prediction of future low awareness periods (step 214) and then observing whether the predicted future low awareness periods actually turned out to be actual low awareness periods when step 216 was performed. Thus, as shown in FIG. 10A, in step 214 three predicted future low awareness periods 116a, 116b, 116c may be determined. When step 216 was then performed and the second signal was transmitted by the controller 50 to initiate the automatic mode, it may also be manually observed whether the predicted future low awareness periods 116a, 116b, 116c turned out to be actual low awareness periods. The determination of whether the predicted future low awareness periods turned out to be actual low awareness periods may be based on observing the sensor data from the sensor 46 during those time periods. If the sensor data is less than a threshold value (e.g., 10 lux for light sensor data) then it may be determined that the predicted future low awareness periods turned out to be an actual low awareness period and thus the method 100 accurately predicted the low awareness period. Similarly, when step 216 is performed, if the sensor data is greater than the threshold value (e.g., 10 lux for light sensor data) then it may be determined that the predicted future low awareness periods turned out not to be an actual low awareness period and thus the method 200 did not accurately predict the low awareness period.

Similar data was obtained for the method 200 predicting a future high awareness period (e.g., for the pesticide dispensing device) and observing whether the predicted future high awareness period turned out to be an actual high awareness period.

Table 2 below shows the four potential outcomes of the above comparison of the predicted future low awareness period(s) and predicted high awareness period(s) with the sensor data that may be used to determine whether each prediction was accurate.

TABLE 2

| True Positive (TP) | False Negative (FN) |
|---|---|
| Light label, matches | Light label, but predicted |
| predicted (1) | is dark (0) |
| False Positive (FP) | True Negative (TN) |
| Dark label, but predicted | Dark label, matches |
| is light (1) | predicted (0) |

The True Positive (TP) result may be based on a predicted future high awareness period in step 214 which turned out to be an actual high awareness period. The False Positive (FP) result may be based on a predicted future high awareness period in step 214 which turned out not to be an actual high awareness period.

The True Negative (TN) result may be based on a predicted future low awareness period in step 214 which turned out to be an actual low awareness period. The False Negative (FN) result may be based on a predicted future low awareness period in step 214 which turned out not to be an actual low awareness period.

The collected data may include a total number of each of TP, FP, TN and FN outcomes, as well as a total number of outcomes.

Based on this collected data, an F1 score was calculated based on the follow equations:

$$F 1 = \frac{2 \times Precision \times Recall}{Prec i s i o n + R e c a l l}$$

where Recall is defined as:

$$Recall = \frac{T P}{T P + F P}$$

and where Precision is defined as:

$$Precision = \frac{T P}{T P + F N}$$

In one example, the collected data resulted in an average Precision of about 0.94, an average Recall of about 0.95 and a weighted average F1 score of about 0.94.

In another example, the collected data resulted in an F1 score of about 0.86. Thus, in some examples, the F1 score of the method 200 disclosed herein may have a value of at least about 0.60, at least about 0.65, at least about 0.7, at least about 0.75, at least about 0.80, at least about 0.85. at least about 0.90 and/or at least about 0.94.

In another example, data was collected using a dataset of 75 homes. In this example, it was found that 9 of the homes experienced sensor malfunctions and thus only 66 homes in the dataset were retained in computing the F1 score. Since the F1 score is calculated for each home, in this example a median value is provided below per home for each of the TP, FP, FN, TN and F1 score. Table 3 below provides these median values for a full data set that considers performance of the model in all time periods of the data and prior to the trapdoor exit decisions are performed (e.g., prior to executing steps 206 through 210 of the method 200).

TABLE 3

| | |
|---|---|
| F1 Median | 0.8593 |
| FP Median | 54.0000 |
| FN Median | 39.0000 |
| TP Median | 319.0000 |
| TN Median | 603.5000 |

Table 4 below provides these median values for a reduced data set that considers the performance of the model in all remaining time periods of the data, after the trapdoor exit decisions are performed (e.g., after execution of steps 206 through 210 of the method 200):

TABLE 4

| | |
|---|---|
| F1 Median | 0.8754 |
| FP Median | 36.0000 |
| FN Median | 30.5000 |
| TP Median | 253.5000 |
| TN Median | 495.5000 |

Additionally, in yet another example, in addition to the above discussed F1 score, an equally or more useful statistic may be a midpoint error, defined as the error in a true value versus a predicted value of the midpoint time of the future low awareness period. In the above discussed example dataset, the value of the mid-point error median for the remaining time period (after execution of the trapdoor exit decisions of steps 206 through 210 are performed) was about 1.1985 hours.

### Aspects

A. A method for operating a device comprising:
   receiving, at a processor, first data over a first time period from a sensor positioned in an area;
   determining, with the processor, second data that indicates an awareness pattern in the area over the first time period based on the first data, said awareness pattern comprising one or more low awareness periods with a low level of awareness in the area and one or more high awareness periods with a high level of awareness in the area;
   determining, with the processor, third data that indicates a value of a characteristic of the first data or the awareness pattern;
   determining, with the processor, whether the value of the characteristic is within a predetermined value range;
   when the value of the characteristic is not within the predetermined value range transmitting, from the processor, a first signal to the device such that the device operates in a manual mode; and
   when the value of the characteristic is within the predetermined value range;
      determining, with the processor, a predicted future low awareness period over a second time period after the first time period based on the awareness pattern of the second data, and
      transmitting, from the processor, a second signal to the device such that the device operates in an automatic mode during the predicted future low awareness period.
B. The method of paragraph A, wherein the device is selected from the group comprising an air freshening device, an air treatment device and a pesticide dispensing device.
C. The method of any of paragraphs A or B, wherein the sensor is a light sensor and the receiving step comprises receiving, at the processor, the first data that comprises light sensor data over the first time period from the light sensor positioned in the area.
D. The method of any of paragraphs A to C, wherein the characteristic is one or more of:
   a) a level of continuity of the first data over the first time period;
   b) a difference between a maximum value and a minimum value of the first data over the first time period;
   c) a difference between a first distance between a first pair of consecutive low awareness periods of the awareness pattern and a second distance between a second pair of consecutive low awareness periods of the awareness pattern;
   d) a duration of one of the low awareness periods or the high awareness periods; and
   e) a number of the one or more low awareness periods or the one or more high awareness periods within a fixed time interval.
E. The method of any of paragraphs A to D, wherein the characteristic comprises two or more of a) to e).
F. The method of any of paragraphs A to E, wherein the characteristic comprises three or more of a) to e).
G. The method of any of paragraphs A to F, wherein the characteristic comprises each of a) to e).
H. The method of any of paragraphs A to G, wherein the determining the second data comprises suppressing, with the processor, values of the first data that exceed a first data threshold.
I. The method of any of paragraphs A to H, wherein the sensor is a light sensor, the first data is light sensor data and the first data threshold is in a range from about 3 lux to about 4000 lux.
J. The method of any of paragraphs A to I, wherein the determining the second data comprises modifying, with the processor, the first data over the first time period to obtain a smoothed curve based on the first data over the first time period and wherein the determining the second data is based on the smoothed curve.
K1. The method of any of paragraphs A to J, wherein the determining the smoothed curve further comprises digitizing the smoothed curve.
K2. The method of any of paragraphs A to J, wherein the determining the smoothed curve further comprises normalizing a value of the smoothed curve to obtain normalized data and digitizing the normalized data.
K3. The method of any of paragraphs A to I, wherein the determining the second data comprises modifying, with the processor, normalizing the first data over the first time period to obtain normalized data and digitizing the normalized data to obtain digitized data, and wherein the determining the second data is based on the digitized data.
K4. The method of any of paragraphs A to I, wherein the determining the second data comprises modifying, with the processor, digitizing the first data over the first time period to obtain digitized data, and wherein the determining the second data is based on the digitized data.
L. The method of any of paragraphs A to K4, wherein the characteristic comprises at least c) and wherein the difference is a standard deviation between the first distance between centers of the first pair of consecutive low awareness periods and the second distance between centers of the second pair of consecutive low awareness periods.
M. The method of any of paragraphs A to L, wherein the characteristic comprises at least d) and wherein the predetermined value range comprises a predetermined low threshold duration that is about 3 hours and a predetermined high threshold duration that is about 18 hours.
N. The method of any of paragraphs A to M, wherein the characteristic comprises at least e) and wherein the fixed time interval is between about 12 hours and about 3 days.
O. The method of any of paragraphs A to N, wherein the determining the predicted future low awareness period comprises predicting a midpoint of the predicted future low awareness period and wherein the transmitting the second signal to the device is such that the device commences operation in the automatic mode at the midpoint of the predicted future low awareness period.
P. The method of any of paragraphs A to O, wherein the transmitting the second signal to the device is such that the device commences operation in the automatic mode prior to commencement of the predicted future low awareness period based on a value of the one of more low awareness periods of the awareness pattern being less than a low duration threshold.
Q. The method of any of paragraphs A to P, further comprising:
   operating the device in the automatic mode during the predicted future low awareness period based on receiving the second signal from the processor; and
   operating the device in the manual mode based on the receiving the first signal from the processor.
R. A method for operating a device, comprising:
   receiving, at a processor, first data over a first time period from a light sensor positioned in an area;
   determining, with the processor, second data that indicates an awareness pattern in the area over the first time period based on the first data, said awareness pattern comprising one or more low awareness periods with a low level of awareness in the area and one or more high awareness periods with a high level of awareness in the area;
   determining, with the processor, third data that indicates a value of a characteristic of the first data or the awareness pattern, wherein the characteristic is one or more of;
      a) a level of continuity of the first data over the first time period,
      b) a difference between a maximum value and a minimum value of the first data over the first time period,
      c) a difference between a first distance between a first pair of consecutive low awareness periods of the awareness pattern and a second distance between a second pair of consecutive low awareness periods of the awareness pattern,
      d) a duration of one of the low awareness periods or the high awareness periods, and
      e) a number of the one or more low awareness periods or the one or more high awareness periods within a fixed time interval;
   determining, with the processor, whether the value of the characteristic is within a predetermined value range;
   when the value of the characteristic is not within the predetermined value range transmitting, from the processor, a first signal to the device such that the device operates in a manual mode; and
   when the value of the characteristic is within the predetermined value range;
      determining, with the processor, a predicted future low awareness period over a second time period after the first time period based on the awareness pattern of the second data, and
      transmitting, from the processor, a second signal to the device such that the device operates in an automatic mode during the predicted future low awareness period.
S. A system comprising:
   a device;
   a sensor positioned in an area and configured to measure first data;
   a processor communicatively coupled with the sensor and the device and configured to receive the first data from the sensor over a first time period;
   wherein the processor is configured to determine second data that indicates an awareness pattern in the area over the first time period based on the first data, said awareness pattern comprising one or more low awareness periods with a low level of awareness in the area and one or more high awareness periods with a high level of awareness in the area;
   wherein the processor is configured to determine third data that indicates a value of a characteristic of the first data or the awareness pattern;
   wherein the processor is configured to determine whether the value of the characteristic is within a predetermined value range;
   when the value of the characteristic is not within the predetermined value range, the processor is configured to transmit a first signal to the device such that the device operates in a manual mode;
   when the value of the characteristic is within the predetermined value range, the processor is configured to:
      determine a predicted future low awareness period over a second time period after the first time period based on the awareness pattern of the second data, and
      transmit a second signal to the device such that the device operates in an automatic mode during the predicted future low awareness period.
T. The system of paragraph S, wherein the device is selected from the group comprising an air freshening device, an air treatment device and a pesticide dispensing device.
U. The system of paragraph S or T, wherein the sensor is a light sensor and the processor is configured to receive the first data that is light sensor data over the first time period from the light sensor positioned in the area.
V. The system of any of paragraphs S to U, wherein the device comprises a reservoir including a volatile composition, a delivery engine in contact with the volatile composition and an evaporative assistance element operatively connected to the delivery engine and configured to adjust a temperature of the fluid in contact with the delivery engine;
   wherein when the value of the characteristic is within the predetermined value range the processor is configured to transmit the second signal to the evaporative assistance element to cause the evaporative assistance element to adjust the temperature of the fluid in contact with the delivery engine to a low temperature in the automatic mode during the predicted future low awareness period; and
   wherein when the value of the characteristic is not within the predetermined value range the processor is configured to transmit the first signal to the evaporative assistance element to cause the evaporative assistance element to adjust the temperature of the fluid in contact with the delivery engine to a high temperature that is greater than the low temperature during the manual mode.
W. The system of any of paragraphs S to V, wherein the characteristic is one or more of:
   a) a level of continuity of the first data over the first time period;
   b) a difference between a maximum value and a minimum value of the first data over the first time period;
   c) a difference between a first distance between a first pair of consecutive low awareness periods of the awareness pattern and a second distance between a second pair of consecutive low awareness periods of the awareness pattern;
   d) a duration of one of the low awareness periods or the high awareness periods; and
   e) a number of the one or more low awareness periods or the one or more high awareness periods within a fixed time interval.
X. The system of any of paragraphs S to W, wherein the characteristic comprises two or more of a) to e).
Y. The system of any of paragraphs S to X, wherein the characteristic comprises three or more of a) to e).
Z. The system of any of paragraphs S to Y, wherein the characteristic comprises each of a) to e).
AA. The method of any of paragraphs S to Z, further comprising:
   determining, with the processor, whether a value of the first data over the first time period is within a predetermined value range of the first data; and
   transmitting, from the processor, a third signal to the device during the first time period such that the device operates in one of the automatic mode or the manual mode during the first time period based on the determining step.
AB. The method of any of paragraphs S to AA,
   wherein the determining step comprises determining, with the processor, whether the value of the first data is less than a first threshold value of the first data over an incremental time period within the first time period; and
   wherein the transmitting step comprises transmitting, from the processor, the third signal to the device during the first time period such that the device operates during the first time period in the automatic mode based on the determining step.
AC. The method of any of paragraphs S to AB, wherein the sensor is a light sensor, wherein the first threshold value is between about 1 lux and about 10 lux, the incremental time period is between about 15 seconds and about 10 days and the first time period is between about 1 day and about 30 days.
AD. The method of any of paragraphs S to AC, wherein the transmitting the third signal to the device is configured such that the device operates in the automatic mode during the first time period for no more than a maximum threshold time period.
AE. The method of any of paragraphs S to AD, wherein the maximum threshold time period is between about 1 hour and about 20 days and wherein the first time period is between about 1 day and about 30 days.
AF. The method of any of paragraphs S to AE,
   wherein the determining step comprises determining, with the processor, whether the value of the first data is greater than a second threshold value of the first data over an incremental time period within the first time period; and
   wherein the transmitting step comprises transmitting, from the processor, the third signal to the device during the first time period such that the device operates in the manual mode based on the determining step.
AG. The method of any of paragraphs S to AF, wherein the sensor is a light sensor, wherein the second threshold value is between about 1 lux and about 10 lux, the incremental time period is between about 15 seconds and about 20 days and wherein the first time period is between about 1 day and about 30 days.
AH. A method for operating the device comprising:
   operating the device in a first state, and
   operating the device according to the method of any of paragraphs A to Q
   wherein operating the device in the first state comprises:
      receiving, at the processor, initial first data from the sensor;
      determining, with the processor, initial second data that indicates an awareness level in the area, said awareness level comprising a low level of current awareness in the area or a high level of current awareness in the area; and
      transmitting an initial signal from the processor to the device such that the device operates in the automatic mode during a low level of current awareness.

### Hardware

FIG. 11 is a block diagram that illustrates a computer system 300 upon which an embodiment of the invention may be implemented. Computer system 300 includes a communication mechanism such as a bus 310 for passing information between other internal and external components of the computer system 300. Information may be represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena may represent digits of a higher base. A superposition of multiple simultaneous quantum states before measurement represents a quantum bit (qubit). A sequence of one or more digits constitutes digital data that may be used to represent a number or code for a character. In some embodiments, information called analog data may be represented by a near continuum of measurable values within a particular range. Computer system 300, or a portion thereof, constitutes a means for performing one or more steps of one or more methods described herein.

A sequence of binary digits constitutes digital data that may be used to represent a number or code for a character. A bus 310 includes many parallel conductors of information so that information may be transferred quickly among devices coupled to the bus 310. One or more processors 302 for processing information are coupled with the bus 310. A processor 302 performs a set of operations on information. The set of operations include bringing information in from the bus 310 and placing information on the bus 310. The set of operations also typically include comparing two or more units of information, shifting positions of units of information, and combining two or more units of information, such as by addition or multiplication. A sequence of operations to be executed by the processor 302 constitutes computer instructions.

Computer system 300 also includes a memory 304 coupled to bus 310. The memory 304, such as a random access memory (RAM) or other dynamic storage device, stores information including computer instructions. Dynamic memory allows information stored therein to be changed by the computer system 300. RAM allows a unit of information stored at a location called a memory address to be stored and retrieved independently of information at neighboring addresses. The memory 304 may be also used by the processor 302 to store temporary values during execution of computer instructions. The computer system 300 also includes a read only memory (ROM) 306 or other static storage device coupled to the bus 310 for storing static information, including instructions, that may not be changed by the computer system 300. Also coupled to bus 310 may be a non-volatile (persistent) storage device 308, such as a magnetic disk or optical disk, for storing information, including instructions, that persists even when the computer system 300 may be turned off or otherwise loses power.

Information, including instructions, may be provided to the bus 310 for use by the processor from an external input device 312, such as a keyboard containing alphanumeric keys operated by a human user, or a sensor. A sensor detects conditions in its vicinity and transforms those detections into signals compatible with the signals used to represent information in computer system 300. Other external devices coupled to bus 310, used primarily for interacting with humans, include a display device 314, such as a cathode ray tube (CRT) or a liquid crystal display (LCD), for presenting images, and a pointing device 316, such as a mouse or a trackball or cursor direction keys, for controlling a position of a small cursor image presented on the display 314 and issuing commands associated with graphical elements presented on the display 314.

In the illustrated embodiment, special purpose hardware, such as an application specific integrated circuit (IC) 320, may be coupled to bus 310. The special purpose hardware may be configured to perform operations not performed by processor 302 quickly enough for special purposes. Examples of application specific ICs include graphics accelerator cards for generating images for display 314, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 300 also includes one or more instances of a communications interface 370 coupled to bus 310. Communication interface 370 provides a two-way communication coupling to a variety of external devices that operate with their own processors, such as printers, scanners, and external disks. In general, the coupling may be with a network link 378 that may be connected to a local network 380 to which a variety of external devices with their own processors are connected. For example, communication interface 370 may be a parallel port or a serial port or a universal serial bus (USB) port on a personal computer. In some embodiments, communications interface 370 may be an integrated services digital network (ISDN) card or a digital subscriber line (DSL) card or a telephone modem that provides an information communication connection to a corresponding type of telephone line. In some embodiments, a communication interface 370 may be a cable modem that converts signals on bus 310 into signals for a communication connection over a coaxial cable or into optical signals for a communication connection over a fiber optic cable. As another example, communications interface 370 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization, or other physical properties of carrier waves. For wireless links, the communications interface 370 sends and receives electrical, acoustic, or electromagnetic signals, including infrared and optical signals, that carry information streams, such as digital data.

The term computer-readable medium may be used herein to refer to any medium that participates in providing information to processor 302, including instructions for execution. Such a medium may take many forms, including, but not limited to, non-volatile media, volatile media, and transmission media. Non-volatile media include, for example, optical or magnetic disks, such as storage device 308. Volatile media include, for example, dynamic memory 304. Transmission media include, for example, coaxial cables, copper wire, fiber optic cables, and waves that travel through space without wires or cables, such as acoustic waves and electromagnetic waves, including radio, optical and infrared waves. The term computer-readable storage medium may be used herein to refer to any medium that participates in providing information to processor 302, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer may read. The term non-transitory computer-readable storage medium may be used herein to refer to any medium that participates in providing information to processor 302, except for carrier waves and other signals.

Logic encoded in one or more tangible media includes one or both of processor instructions on a computer-readable storage media and special purpose hardware, such as ASIC *320.

Network link 378 typically provides information communication through one or more networks to other devices that use or process the information. For example, network link 378 may provide a connection through local network 380 to a host computer 382 or to equipment 384 operated by an Internet Service Provider (ISP). ISP equipment 384 in turn provides data communication services through the public, world-wide packet-switching communication network of networks now commonly referred to as the Internet 390. A computer called a server 392 connected to the Internet provides a service in response to information received over the Internet. For example, server 392 provides information representing video data for presentation at display 314.

The disclosure may be related to the use of computer system 300 for implementing the techniques described herein. According to one embodiment of the invention, those techniques are performed by computer system 300 in response to processor 302 executing one or more sequences of one or more instructions contained in memory 304. Such instructions, also called software and program code, may be read into memory 304 from another computer-readable medium such as storage device 308. Execution of the sequences of instructions contained in memory 304 causes processor 302 to perform the method steps described herein. In alternative embodiments, hardware, such as application specific integrated circuit 320, may be used in place of or in combination with software to implement the invention. Thus, embodiments of the invention are not limited to any specific combination of hardware and software.

The signals transmitted over network link 378 and other networks through communications interface 370, carry information to and from computer system 300. Computer system 300 may send and receive information, including program code, through the networks 380, 390 among others, through network link 378 and communications interface 370. In an example using the Internet 390, a server 392 transmits program code for a particular application, requested by a message sent from computer 300, through Internet 390, ISP equipment 384, local network 380 and communications interface 370. The received code may be executed by processor 302 as it may be received or may be stored in storage device 308 or other non-volatile storage for later execution, or both. In this manner, computer system 300 may obtain application program code in the form of a signal on a carrier wave.

Various forms of computer readable media may be involved in carrying one or more sequence of instructions or data or both to processor 302 for execution. For example, instructions and data may initially be carried on a magnetic disk of a remote computer such as host 382. The remote computer loads the instructions and data into its dynamic memory and sends the instructions and data over a telephone line using a modem. A modem local to the computer system 300 receives the instructions and data on a telephone line and uses an infra-red transmitter to convert the instructions and data to a signal on an infra-red a carrier wave serving as the network link 378. An infrared detector serving as communications interface 370 receives the instructions and data carried in the infrared signal and places information representing the instructions and data onto bus 310. Bus 310 carries the information to memory 304 from which processor 302 retrieves and executes the instructions using some of the data sent with the instructions. The instructions and data received in memory 304 may optionally be stored on storage device 308, either before or after execution by the processor 302.

FIG. 12 illustrates a chip set 400 upon which an embodiment of the invention may be implemented. Chip set 400 may be programmed to perform one or more steps of a method described herein and includes, for instance, the processor and memory components described with respect to FIG. 4 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It may be contemplated that in certain embodiments the chip set may be implemented in a single chip. Chip set 400, or a portion thereof, constitutes a means for performing one or more steps of a method described herein.

In one embodiment, the chip set 400 includes a communication mechanism such as a bus 401 for passing information among the components of the chip set 400. A processor 403 has connectivity to the bus 401 to execute instructions and process information stored in, for example, a memory 405. The processor 403 may include one or more processing cores with each core configured to perform independently. A multi-core processor enables multiprocessing within a single physical package. Examples of a multi-core processor include two, four, eight, or greater numbers of processing cores. Alternatively or in addition, the processor 403 may include one or more microprocessors configured in tandem via the bus 401 to enable independent execution of instructions, pipelining, and multithreading. The processor 403 may also be accompanied with one or more specialized components to perform certain processing functions and tasks such as one or more digital signal processors (DSP) 407, or one or more application-specific integrated circuits (ASIC) 409. A DSP 407 typically may be configured to process real-world signals (e.g., sound) in real time independently of the processor 403. Similarly, an ASIC 409 may be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 403 and accompanying components have connectivity to the memory 405 via the bus 401. The memory 405 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 405 also stores the data associated with or generated by the execution of one or more steps of the methods described herein.

FIG. 13 may be a diagram of exemplary components of a mobile terminal 500 (e.g., cell phone handset) for communications, which may be capable of operating in the dispenser of FIG. 4, according to one embodiment. In some embodiments, mobile terminal 501, or a portion thereof, constitutes a means for performing one or more steps described herein. Generally, a radio receiver may be often defined in terms of front-end and back-end characteristics. The front-end of the receiver encompasses all of the Radio Frequency (RF) circuitry whereas the back-end encompasses all of the base-band processing circuitry. As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory(ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 503, a Digital Signal Processor (DSP) 505, and a receiver/transmitter unit including a microphone gain control unit and a speaker gain control unit. A main display unit 507 provides a display to the user in support of various applications and mobile terminal functions that perform or support the steps as described herein. The display 507 includes display circuitry configured to display at least a portion of a user interface of the mobile terminal (e.g., mobile telephone). Additionally, the display 507 and display circuitry are configured to facilitate user control of at least some functions of the mobile terminal. An audio function circuitry 509 includes a microphone 511 and microphone amplifier that amplifies the speech signal output from the microphone 511. The amplified speech signal output from the microphone 511 may be fed to a coder/decoder (CODEC) 513.

A radio section 515 amplifies power and converts frequency in order to communicate with a base station, which may be included in a mobile communication system, via antenna 517. The power amplifier (PA) 519 and the transmitter/modulation circuitry are operationally responsive to the MCU 503, with an output from the PA 519 coupled to the duplexer 521 or circulator or antenna switch, as known in the art. The PA 519 also couples to a battery interface and power control unit 520.

In use, a user of mobile terminal 501 speaks into the microphone 511 and his or her voice along with any detected background noise may be converted into an analog voltage. The analog voltage may be then converted into a digital signal through the Analog to Digital Converter (ADC) 523. The control unit 503 routes the digital signal into the DSP 505 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wideband code division multiple access (WCDMA), wireless fidelity (WiFi), satellite, and the like, or any combination thereof.

The encoded signals are then routed to an equalizer 525 for compensation of any frequency-dependent impairments that occur during transmission though the air such as phase and amplitude distortion. After equalizing the bit stream, the modulator 527 combines the signal with a RF signal generated in the RF interface 529. The modulator 527 generates a sine wave by way of frequency or phase modulation. In order to prepare the signal for transmission, an up-converter 531 combines the sine wave output from the modulator 527 with another sine wave generated by a synthesizer 533 to achieve the desired frequency of transmission. The signal may be then sent through a PA 519 to increase the signal to an appropriate power level. In practical systems, the PA 519 acts as a variable gain amplifier whose gain may be controlled by the DSP 505 from information received from a network base station. The signal may be then filtered within the duplexer 521 and optionally sent to an antenna coupler 535 to match impedances to provide maximum power transfer. Finally, the signal may be transmitted via antenna 517 to a local base station. An automatic gain control (AGC) may be supplied to control the gain of the final stages of the receiver. The signals may be forwarded from there to a remote telephone which may be another cellular telephone, any other mobile phone or a land-line connected to a Public Switched Telephone Network (PSTN), or other telephony networks.

Voice signals transmitted to the mobile terminal 501 are received via antenna 517 and immediately amplified by a low noise amplifier (LNA) 537. A down-converter 539 lowers the carrier frequency while the demodulator 541 strips away the RF leaving only a digital bit stream. The signal then goes through the equalizer 525 and may be processed by the DSP 505. A Digital to Analog Converter (DAC) 543 converts the signal and the resulting output may be transmitted to the user through the speaker 545, all under control of a Main Control Unit (MCU) 503 which may be implemented as a Central Processing Unit (CPU) (not shown).

The MCU 503 receives various signals including input signals from the keyboard 547. The keyboard 547 and/or the MCU 503 in combination with other user input components (e.g., the microphone 511) comprise a user interface circuitry for managing user input. The MCU 503 runs a user interface software to facilitate user control of at least some functions of the mobile terminal 501 as described herein. The MCU 503 also delivers a display command and a switch command to the display 507 and to the speech output switching controller, respectively. Further, the MCU 503 exchanges information with the DSP 505 and may access an optionally incorporated SIM card 549 and a memory 551. In addition, the MCU 503 executes various control functions required of the terminal. The DSP 505 may, depending upon the implementation, perform any of a variety of conventional digital processing functions on the voice signals. Additionally, DSP 505 determines the background noise level of the local environment from the signals detected by microphone 511 and sets the gain of microphone 511 to a level selected to compensate for the natural tendency of the user of the mobile terminal 501.

The CODEC 513 includes the ADC 523 and DAC 543. The memory 551 stores various data including call incoming tone data and may be capable of storing other data including music data received via, e.g., the global Internet. The software module could reside in RAM memory, flash memory, registers, or any other form of writable storage medium known in the art. The memory device 551 may be, but not limited to, a single memory, CD, DVD, ROM, RAM, EEPROM, optical storage, magnetic disk storage, flash memory storage, or any other non-volatile storage medium capable of storing digital data.

An optionally incorporated SIM card 549 carries, for instance, important information, such as the cellular phone number, the carrier supplying service, subscription details, and security information. The SIM card 549 serves primarily to identify the mobile terminal 501 on a radio network. The card 549 also contains a memory for storing a personal telephone number registry, text messages, and user specific mobile terminal settings.

In some embodiments, the mobile terminal 501 includes a digital camera comprising an array of optical detectors, such as charge coupled device (CCD) array 565. The output of the array may be image data that may be transferred to the MCU for further processing or storage in the memory 551 or both. In the illustrated embodiment, the light impinges on the optical array through a lens 563, such as a pin-hole lens or a material lens made of an optical grade glass or plastic material. In the illustrated embodiment, the mobile terminal 501 includes a light source 561, such as a LED to illuminate a subject for capture by the optical array, e.g., CCD 565. The light source may be powered by the battery interface and power control module 520 and controlled by the MCU 503 based on instructions stored or loaded into the MCU 503.

As used herein, the word “comprising” may be interpreted as requiring the features mentioned, but not limiting the presence of other features. Alternatively, the word “comprising” may also relate to the situation where only the components/features listed are intended to be present (e.g., the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

As used in this specification and the appended claims, the singular forms “a”, “an”, and “the” include plural references unless the content clearly dictates otherwise. Thus, for example, “a volatile material” may include more than one volatile material.

The dimensions and values disclosed herein are not to be understood as being strictly limited to the exact numerical values recited. Furthermore, dimensions should not be held to an impossibly high standard of metaphysical identity that does not allow for discrepancies due to typical manufacturing tolerances. Therefore, the term “about” should be interpreted as being within typical manufacturing and measuring tolerances.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method for operating a device, comprising:
receiving, at a processor, first data over a first time period from a sensor positioned in an area;
determining, with the processor, second data that indicates an awareness pattern in the area over the first time period based on the first data, said awareness pattern comprising one or more low awareness periods with a low level of awareness in the area and one or more high awareness periods with a high level of awareness in the area;
determining, with the processor, third data that indicates a value of a characteristic of the first data or the awareness pattern; and
determining, with the processor, whether the value of the characteristic is within a predetermined value range;
when the value of the characteristic is not within the predetermined value range, transmitting a first signal from the processor to the device such that the device operates in a manual mode; and
when the value of the characteristic is within the predetermined value range:
determining, with the processor, a predicted future low awareness period over a second time period after the first time period based on the awareness pattern of the second data, and
transmitting a second signal from the processor to the device such that the device operates in an automatic mode during the predicted future low awareness period.

2. The method of claim 1, wherein the device is selected from the group consisting of an air freshening device, an air treatment device, and a pesticide dispensing device.

3. The method of claim 1, wherein the sensor comprises a light sensor and the receiving step comprises receiving, at the processor, the first data that comprises light sensor data over the first time period from the light sensor positioned in the area.

4. The method of claim 1, wherein the characteristic is one or more of:
a) a level of continuity of the first data over the first time period;
b) a difference between a maximum value and a minimum value of the first data over the first time period;
c) a difference between a first distance between a first pair of consecutive low awareness periods or consecutive high awareness periods of the awareness pattern and a second distance between a second pair of consecutive low awareness periods or consecutive high awareness periods of the awareness pattern;
d) a duration of one of the low awareness periods or the high awareness periods; and
e) a number of transitions between the one or more low awareness periods and the one or more high awareness periods within a fixed time interval.

5. The method of claim 4, wherein the characteristic comprises four or more of a) to e).

6. The method of claim 4, wherein the characteristic comprises at least c) and wherein the difference is a standard deviation between the first distance between centers of the first pair of consecutive low awareness periods or consecutive high awareness periods and the second distance between centers of the second pair of consecutive low awareness periods or consecutive high awareness periods.

7. The method of claim 4, wherein the characteristic comprises at least d) and wherein the predetermined value range comprises a predetermined low threshold duration that is about 3 hours and a predetermined high threshold duration that is about 18 hours.

8. The method of claim 4, wherein the characteristic comprises at least e) and wherein the fixed time interval is between about 12 hours and about 3 days, and the number is at least 2.

9. The method of claim 1, wherein the determining the second data comprises suppressing, with the processor, values of the first data that exceed a first data threshold.

10. The method of claim 9, wherein the sensor is a light sensor, the first data is light sensor data, and the first data threshold is in a range from about 3 lux to about 4000 lux.

11. The method of claim 1, wherein the determining the second data comprises modifying, with the processor, the first data over the first time period to obtain a smoothed curve based on the first data over the first time period, and wherein the determining the second data is based on the smoothed curve.

12. The method of claim 11, wherein the determining the smoothed curve further comprises normalizing a value of the smoothed curve and digitizing the smoothed curve.

13. The method of claim 1, wherein the determining the predicted future low awareness period comprises predicting a midpoint of the predicted future low awareness period and wherein the transmitting the second signal to the device is such that the device commences operation in the automatic mode at the midpoint of the predicted future low awareness period.

14. The method of claim 1, wherein the transmitting the second signal to the device is such that the device commences operation in the automatic mode prior to commencement of the predicted future low awareness period based on a value of the one of more low awareness periods of the awareness pattern being less than a low duration threshold.

15. The method of claim 1, further comprising:
operating the device in the automatic mode during the predicted future low awareness period based on receiving the second signal from the processor; and
operating the device in the manual mode based on the receiving the first signal from the processor.

16. The method of claim 1, further comprising:
determining, with the processor, whether a value of the first data over the first time period is within a predetermined value range of the first data; and
transmitting, from the processor, a third signal to the device during the first time period such that the device operates in one of the automatic mode or the manual mode during the first time period based on the determining step,
wherein the determining step comprises determining, with the processor, whether the value of the first data is less than a first threshold value of the first data over an incremental time period within the first time period, and
wherein the transmitting step comprises transmitting, from the processor, the third signal to the device during the first time period such that the device operates during the first time period in the automatic mode based on the determining step.

17. The method of claim 16, wherein the sensor is a light sensor, wherein the first threshold value is between about 1 lux and about 10 lux, the incremental time period is between about 15 seconds and about 10 days and the first time period is between about 1 day and about 30 days.

18. The method of claim 16, wherein the transmitting the third signal to the device is configured such that the device operates in the automatic mode during the first time period for no more than a maximum threshold time period.

19. The method of claim 18, wherein the maximum threshold time period is between about 1 hour and about 20 days and wherein the first time period is between about 1 day and about 30 days.

20. The method of claim 19, wherein the determining step comprises determining, with the processor, whether the value of the first data is greater than a second threshold value of the first data over an incremental time period within the first time period; and
wherein the transmitting step comprises transmitting, from the processor, the third signal to the device during the first time period such that the device operates in the manual mode based on the determining step.

21. A system comprising:
a device;
a sensor positioned in an area and configured to measure first data; and
a processor communicatively coupled with the sensor and the device and configured to receive the first data from the sensor over a first time period;
wherein the processor is configured to determine second data that indicates an awareness pattern in the area over the first time period based on the first data, said awareness pattern comprising one or more low awareness periods with a low level of awareness in the area and one or more high awareness periods with a high level of awareness in the area;
wherein the processor is configured to determine third data that indicates a value of a characteristic of the first data or the awareness pattern;
wherein the processor is configured to determine whether the value of the characteristic is within a predetermined value range; and
wherein:
when the value of the characteristic is not within the predetermined value range, the processor is configured to transmit a first signal to the device such that the device operates in a manual mode; and
when the value of the characteristic is within the predetermined value range, the processor is configured to:
determine a predicted future low awareness period over a second time period after the first time period based on the awareness pattern of the second data, and
transmit a second signal to the device such that the device operates in an automatic mode during the predicted future low awareness period.
